(19) United States
(12) Patent Application Publication
(10) Pub. No.: US 2026/0114751 A1
(43) Pub. Date: Apr. 30, 2026

(54) POSTURE DETECTION AND PATIENT MANAGEMENT

(71) Applicant: Cardiac Pacemakers, Inc., St. Paul, MN (US)

(72) Inventors: Pushti DESAI, North Wales, PA (US); Pramodsingh Hirasingh THAKUR, Woodbury, MN (US); Viktoria A. AVERINA, Shoreview, MN (US); Jonathan Bennett SHUTE, Eagan, MN (US)

(21) Appl. No.: 19/364,770

(22) Filed: Oct. 21, 2025

Related U.S. Application Data
(60) Provisional application No. 63/713,144, filed on Oct. 29, 2024.

(51) Int. Cl. A61B 5/11; A61B 5/00

(52) U.S. Cl. CPC A61B 5/1116; A61B 5/7253; A61B 5/7264; A61B 5/7275; A61B 5/742; A61B 5/746; A61B 2562/0219

(57) ABSTRACT

Systems and methods are disclosed for improving posture detection to optimize medical device system resource allocation, including clustering received posture information occurring over a first time period into at least two clusters including a supine or upright cluster and at least one other cluster, determining, for each respective cluster, an opposite density measure indicative of a density of the posture information opposite the respective cluster in multi-dimensional space with respect to the posture sensor, identifying the supine or upright cluster using the opposite density measures of the at least two clusters, and determining the posture information for the patient using the identified supine or upright cluster.

### CROSS REFERENCE TO RELATED APPLICATION

This application claims the benefit of and priority to U.S. Provisional Application No. 63/713,144, filed Oct. 29, 2024, the entire contents of which is hereby incorporated by reference.

### TECHNICAL FIELD

This document relates generally to medical devices and more particularly to systems and methods for improving posture detection to optimize medical device system resource allocation.

### BACKGROUND

Ambulatory medical devices (AMDs), including implantable, subcutaneous, wearable, insertable, or one or more other medical devices, etc., can monitor, detect, or treat various conditions, including heart failure (HF), arrhythmia, etc. Ambulatory medical devices can include sensors to sense physiologic information from a patient and one or more circuits to detect one or more physiologic events using the sensed physiologic information or transmit sensed physiologic information or detected physiologic events to one or more remote devices. Additionally, ambulatory medical devices can be configured to provide electrical stimulation or one or more other therapies or treatments to the patient, such as to improve cardiac function, etc.

Ambulatory patient monitoring can provide early detection of worsening patient condition, including worsening heart failure, arrhythmia, etc. Accurate identification of patients or groups of patients at an elevated risk of future adverse events may control mode or feature selection or resource management of one or more medical devices, control notifications or messages in connected systems to various users associated with a specific patient or group of patients, organize or schedule physician or patient contact or treatment, or prevent or reduce patient hospitalization. Correctly identifying and safely managing patient risk of worsening condition may avoid unnecessary medical interventions, extend the usable life of medical devices, and reduce healthcare costs. In addition, in situations where different operating modes, features, or therapies are available, correctly monitoring, detecting, and identifying patient health status, including improving or worsening patient condition, and modifying one or more medical device functions based thereon, can improve medical device efficiency, such as by reducing unnecessary resource consumption, thereby extending the usable life of the ambulatory medical device.

### SUMMARY

Systems and methods are disclosed for improving posture detection to optimize medical device system resource allocation, including clustering received posture information occurring over a first time period into at least two clusters including a supine or upright cluster and at least one other cluster, determining, for each respective cluster, an opposite density measure indicative of a density of the posture information opposite the respective cluster in multi-dimensional space with respect to the posture sensor, identifying the supine or upright cluster using the opposite density measures of the at least two clusters, and determining the posture information for the patient using the identified supine or upright cluster.

An example of subject matter (e.g., a medical device system for improving posture detection to optimize medical device system resource allocation) may comprise means for receiving posture information of a patient from a posture sensor of an ambulatory medical device, the posture information including posture information from multiple axes with respect to the posture sensor of the ambulatory medical device means for clustering the received posture information occurring over a first time period into at least two clusters, wherein one of the at least two clusters includes a supine or upright cluster and at least one other cluster means for determining, for each respective cluster, an opposite density measure indicative of a density of the posture information opposite the respective cluster in multi-dimensional space with respect to the posture sensor means for identifying the supine or upright cluster using the opposite density measures of the at least two clusters and means for determining posture information for the patient using the identified supine or upright cluster.

In an example, which may be combined with any one or more examples described herein, the means for receiving posture information includes a signal receiver circuit configured to receive posture information of the patient from the posture sensor of the ambulatory medical device, wherein the means for clustering, determining, identifying, and determining includes an assessment circuit configured to cluster the received posture information occurring over the first time period into the at least two clusters, wherein one of the at least two clusters includes the supine or upright cluster and the at least one other cluster determine, for each respective cluster, the opposite density measure indicative of the density of the posture information opposite the respective cluster in multi-dimensional space with respect to the posture sensor identify the supine or upright cluster using the opposite density measures of the at least two clusters and determine the posture information for the patient using the identified supine or upright cluster.

An example of subject matter (e.g., a medical device system for improving posture detection to optimize medical device system resource allocation) may comprise a signal receiver circuit configured to receive posture information of a patient from a posture sensor of an ambulatory medical device, the posture information including posture information from multiple axes with respect to the posture sensor of the ambulatory medical device and an assessment circuit configured to determine posture information for the patient using the received posture information, including to cluster the received posture information occurring over a first time period into at least two clusters, wherein one of the at least two clusters includes a supine or upright cluster and at least one other cluster determine, for each respective cluster, an opposite density measure indicative of a density of the posture information opposite the respective cluster in multi-dimensional space with respect to the posture sensor identify the supine or upright cluster using the opposite density measures of the at least two clusters and determine the posture information for the patient using the identified supine or upright cluster.

In an example, which may be combined with any one or more examples described herein, the supine or upright cluster includes a supine/upright cluster, wherein the assessment circuit is configured to identify the supine/upright cluster as the respective cluster having a lowest of the determined opposite density measure.

In an example, which may be combined with any one or more examples described herein, the supine or upright cluster includes the upright cluster, wherein the assessment circuit is configured to identify the upright cluster as the respective cluster having a lowest of the determined opposite density measure.

In an example, which may be combined with any one or more examples described herein, the at least one other cluster includes a first side cluster and a second side cluster, wherein the assessment circuit is configured to identify a left side cluster and a right side cluster using the first side cluster, the second side cluster, and the supine or upright cluster.

In an example, which may be combined with any one or more examples described herein, the posture sensor of the ambulatory medical device includes a three-dimensional accelerometer, wherein the posture information includes three-dimensional acceleration information with respect to the accelerometer of the ambulatory medical device, wherein the assessment circuit is configured to determine, for each respective cluster, the opposite density measure indicative of the density of the three-dimensional acceleration information occurring over the first time period opposite the respective cluster.

In an example, which may be combined with any one or more examples described herein, the assessment circuit is configured to separate the supine or upright cluster into separate supine and upright clusters based on a spread of the three-dimensional acceleration information in the supine or upright cluster, wherein the spread of the supine cluster is less than the spread of the upright cluster.

In an example, which may be combined with any one or more examples described herein, the three-dimensional acceleration information is with respect to UVW space of the accelerometer of the ambulatory medical device, wherein the assessment circuit is configured to determine centroids of at least three clusters and transform the three-dimensional acceleration information from the UVW space of the accelerometer into XYZ space of the patient using the determined centroids of the at least three clusters.

In an example, which may be combined with any one or more examples described herein, to determine the centroids of the at least three clusters includes to determine centroids of the supine cluster, a first or left side cluster, and a second or right side cluster.

In an example, which may be combined with any one or more examples described herein, the assessment circuit is configured to label the three-dimensional acceleration information and to determine posture trends for the patient over the first time period using the labeled information.

In an example, which may be combined with any one or more examples described herein, the assessment circuit is configured to determine a patient health status using changes in the determined posture trends.

In an example, which may be combined with any one or more examples described herein, the assessment circuit is configured to provide an output of the determined posture trends to a user interface for display to a user or to another circuit to control or adjust a process or function of the medical device system.

In an example, which may be combined with any one or more examples described herein, the assessment circuit is configured to compare the determined posture trends for the patient over the first time period to determined posture trends for the patient over one or more other time periods and to provide an alert if the determined posture trends for the patient over the first time period exceed the determined posture trends for the patient over the one or more other time periods by more than a threshold amount.

In an example, which may be combined with any one or more examples described herein, the assessment circuit is configured to alter or adjust one or more modes or functions of the ambulatory medical device or the medical device system based on the determined posture trends for the patient over the first time period, wherein the one or more modes or functions includes at least one of: an active state of a sensor of the medical device system, a sampling frequency or resolution of a sensor of the medical device system, an amount of data storage of the ambulatory medical device or the medical device system, or a time or amount of communication of stored information outside of the ambulatory medical device.

An example of subject matter (e.g., a method for improving posture detection to optimize medical device system resource allocation) may comprise receiving acceleration information of a patient from an accelerometer of an ambulatory medical device, the acceleration information including three-dimensional acceleration information with respect to the accelerometer of the ambulatory medical device and determining posture information for the patient using the received acceleration information, including clustering the received acceleration information occurring over a first time period into at least two clusters, wherein one of the at least two clusters includes a supine or upright cluster and at least one other cluster determining, for each respective cluster, an opposite density measure indicative of a density of the three-dimensional acceleration information opposite the respective cluster identifying the supine or upright cluster using the opposite density measures of the at least two clusters and determining the posture information for the patient using the identified supine or upright cluster.

In an example, which may be combined with any one or more examples described herein, the supine or upright cluster includes a supine/upright cluster, wherein identifying the supine or upright cluster includes identifying the supine/upright cluster as the respective cluster having a lowest of the determined opposite density measure.

In an example, which may be combined with any one or more examples described herein, the supine or upright cluster includes the upright cluster, wherein identifying the supine or upright cluster includes identifying the upright cluster as the respective cluster having a lowest of the determined opposite density measure.

In an example, which may be combined with any one or more examples described herein, the at least one other cluster includes a first side cluster and a second side cluster, wherein determining the posture information for the patient includes identifying a left side cluster and a right side cluster using the first side cluster, the second side cluster, and the supine or upright cluster.

In an example, which may be combined with any one or more examples described herein, determining the posture information for the patient includes separating the supine or upright cluster into separate supine and upright clusters based on a spread of the three-dimensional acceleration information in the supine or upright cluster, wherein the spread of the supine cluster is less than the spread of the upright cluster.

In an example, which may be combined with any one or more examples described herein, the three-dimensional acceleration information is with respect to UVW space of the accelerometer of the ambulatory medical device, wherein determining the posture information for the patient includes determining centroids of at least three clusters, including the supine cluster, a first or left side cluster, and a second or right side cluster and transforming the three-dimensional acceleration information from the UVW space of the accelerometer into XYZ space of the patient using the determined centroids of the at least three clusters.

In an example, which may be combined with any one or more examples described herein, determining the posture information for the patient includes labeling the received acceleration information determining posture trends for the patient over the first time period using the labeled information and determining a patient health status using changes in the determined posture trends.

In an example, a system, method, or apparatus may optionally combine any portion or combination of any portion of any one or more of the examples described herein, may optionally combine any portion or combination of any portion of any one or more of the examples described herein to comprise “means for” performing any portion of any one or more of the functions, operations, or methods of the examples described herein, or at least one “non-transitory machine-readable medium” including instructions that, when performed by a machine, cause the machine to perform any portion of any one or more of the functions or methods of the examples described herein.

This summary is intended to provide an overview of subject matter of the present patent application. It is not intended to provide an exclusive or exhaustive explanation of the disclosure. The detailed description is included to provide further information about the present patent application. Other aspects of the disclosure will be apparent to persons skilled in the art upon reading and understanding the following detailed description and viewing the drawings that form a part thereof, each of which are not to be taken in a limiting sense.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 illustrates an example implantable medical device.

FIG. 2 illustrates an example patient posture trend.

FIGS. 3A-3B illustrate initial clusters of received acceleration information and determined centroids of the initial clusters.

FIGS. 4A-4B illustrate example changes in posture distribution over time in control and heart failure patients.

FIG. 5 illustrates an example change in night side bias before a heart failure event.

FIG. 6 illustrates an example change in nighttime tossing percentage before a heart failure event.

FIG. 7 illustrates an example method to detect patient posture using acceleration information of the patient.

FIG. 8 illustrates an example method to optimize medical device system resource allocation using determined patient posture information.

FIG. 9 illustrates an example medical device system.

FIG. 10 illustrates an example patient management system and portions of an environment in which the system may operate.

FIG. 11 illustrates an example implantable medical device system.

FIG. 12 illustrates example implantable medical devices.

FIG. 13 illustrates example remote patient management systems.

FIG. 14 illustrates an example machine upon which any one or more of the techniques discussed herein may perform.

### DETAILED DESCRIPTION

Ambulatory medical devices are devices configured to be implanted in or otherwise positioned on or about patients to monitor physiologic information, such as cardiac electrical, heart sound, respiration, impedance, pressure, physical activity, or other physiologic information or one or more other physiologic parameters of the patient, or to provide electrical stimulation or one or more other therapies or treatments to optimize or control one or more body functions of the patient, such as contractions of a heart, etc. Ambulatory medical devices can include implantable or external (e.g., wearable) devices configured to monitor or provide stimulation to the patient.

Ambulatory medical devices can provide different monitoring, storage, communication, or therapy using different modes, however, with different power and resource requirements and varying effectiveness for different patients. For example, a variety of therapy modalities are available to patients, but not all patients receive the optimal medical device, therapy mode, or therapy parameter settings at first programming. One common reason for suboptimal ambulatory medical device programming is detection or determination of different events, conditions, or indications. Optimal programming depends on, among other things, accurate detection and determination of different events, conditions, or indications. Desired clinical outcomes can include, among others, cardiac capture, selection of pacing or sensing vectors, pacing mode, resource usage associated with communication or transmission of physiologic data from the ambulatory medical device or storage of physiologic data, etc.

Accurate identification of patients at elevated risk of future adverse events is crucial for effective patient management. This information can be used to control device features, manage resources, trigger notifications, organize treatment, and potentially prevent or reduce patient hospitalization. Correctly identifying and managing patient risk can help avoid unnecessary medical interventions, extend the usable life of medical devices, and reduce healthcare costs.

Current methods for monitoring patient health status and adjusting device functionality often rely on patient information detected using ambulatory medical devices, which commonly require scheduled in-clinic follow-up procedures for optimal programming or calibration. These appointments can be burdensome for patients, requiring travel and time commitments. Moreover, the infrequent nature of these follow-ups may result in delayed detection of changes in patient condition or suboptimal device programming, and not all facilities for in-clinic follow-up have the same equipment (e.g., tables for the patient to lie flat, etc.), severely impacting traditional calibration.

One area of particular interest in patient monitoring is the assessment of patient posture. Changes in posture patterns can be indicative of various clinical conditions, including heart failure, sleep disorders, and overall patient wellness. However, existing methods for posture detection using one or more posture sensors in ambulatory medical devices often require manual intervention or guided movement for calibration or resource intensive algorithms for posture detection without calibration and may not account for changes in patient condition or ambulatory medical device or posture sensor movement or other sensor changes over time.

There is a need for improved systems and methods to monitor patient health status, detect changes in condition, and optimize device functionality based on patient-specific information. Additionally, posture directly impacts certain physiologic information, such as heart sounds, heart rate, etc. For example, noise in heart sound information distinctly correlates with patient posture, such that accurate determination of patient posture can improve heart sound sensing, signal acquisition, and determination of physiologic measures or parameters. Such improvements could enhance the efficiency and effectiveness of ambulatory medical devices, potentially leading to better patient outcomes and more efficient use of healthcare resources.

The present inventors have recognized, among other things, systems and methods to detect patient posture and to perform frequent calibration without manual intervention or guided movement to improve posture determination for improved patient monitoring and device management, such as to transition monitoring modes based on changes in posture trends occurring over time or to reduce processing or other medical device system resources associated with posture determination or calibration of posture sensors in implantable or wearable medical devices. While calibration in implantable medical devices is paramount, calibration of wearable medical device data can be even more challenging due to noise or imprecise placement through repeated application or wear, etc. For the same reasons, chronic posture measurement over extended periods of time (e.g., weeks or months) are important metrics measured by implantable medical devices, which have limited processing and power resources, which makes improvements to existing algorithms even more important.

The systems and methods described herein include an automatic calibration algorithm for a posture sensor, such as a three-dimensional accelerometer, that can determine patient posture from an imprecise and often changing implant location, such as subcutaneous or otherwise implanted in a torso of the patient, etc., using local coordinates with respect to the posture sensor. The automatic calibration algorithm can automatically align accelerometer coordinates in a UVW space with ideal body coordinates in XYZ space with respect to the patient, allowing for accurate posture detection without the need for manual calibration procedures or directed motions or movements that result in calibration.

The present inventors have recognized that instead of focusing on identifying the most common postures and orientations, that calibration is most effective with respect to accelerometer data by identifying the least likely postures and orientations of the patient and making determinations based thereon. For example, a first priority in identifying orientation can be in the negative, that off all orientations, patients are least likely to be standing on their heads. A second priority can include that patients are second least likely to be lying on their stomach. In certain examples, a combined supine/upright orientation can be determined as an identified cluster having a lowest opposite density measure.

Accordingly, the automatic calibration algorithm can, in certain examples, require the following steps: (1) observe and record posture information for a period of time over one or more days; (2) organize and cluster the received posture information orientation in three-dimensional space; (3) assign the data set with the most empty space as the orientation having the patient standing on their head; (4) optionally assign the data set with the second most empty space as the patient lying on their stomach; and (5) determine the remaining clusters as most appropriate given the prior assignments and relationships in accordance therewith.

In an example, received acceleration information, such as for a first time period (e.g., a week, etc.) can be clustered into different initial clusters, for example, using an assessment circuit or one or more other circuits, etc. Starting identification of the different clusters with one or two categories (e.g., standing on their head, lying on their stomach, etc.) provides improved calibration performance than starting with more and other categories (e.g., five, ten, etc.). In an example, a difference between daytime and nighttime posture information can be additionally used to distinguish the two least likely postures or orientations (e.g., standing on their head and lying on their stomach), as a transition between daytime and nighttime posture information (with daytime and nighttime determined by time of day, activity, or one or more other methods) can be used to distinguish between lying on their stomach and standing on their head (e.g., by their opposites, noting a shift between an upright and supine or substantially supine posture information from day to night), whereas a lack of transition between daytime and nighttime posture information can be used to indicate a lack of upright posture, such as during times of hospitalization, bedrest, or other immobile, sedentary, or lack of daily activity. In certain examples, daytime and nighttime differences can be used to distinguish or verify supine/upright (or upright) posture identification. Additionally, changes in detected impedance between position shifts can be used to distinguish between or verify identification of upright versus non-upright postures.

In certain examples, features can be extracted from the self-calibrated posture information, such as time spent in specific postures (e.g., upright, lying down on the left side, right side, supine, or prone, etc.), restlessness (e.g., frequency of changing postures), and side bias. These features can be used to monitor and alert for worsening conditions, such as heart failure, COPD, sleep apnea, and general wellness. The posture information can be used to monitor posture-dependent changes in physiologic metrics or device parameters (e.g., cardiac resynchronization therapy (CRT) or conduction system pacing (CSP) when lying on left side vs. right side, etc.), report posture-dependent versions of existing device metrics (e.g., apnea-hypopnea index (AHI) determination at lying down postures only, right side vs. left side, etc.), detect life events (e.g., all-cause hospitalization, travel periods, etc.), or trigger changes in other sensors (e.g., sampling, trigger or stop another algorithm, trigger or stop a communication protocol, etc.), physiologic metrics, parameters, or medical device system functions.

In certain examples, posture trends can be determined and analyzed over time, for example, calculating daily posture percentages, creating heatmaps of posture percentages, or detecting abnormal changes between consecutive days of posture data (e.g., between different time periods, such as short-term and long-term periods, successive time periods, etc.). This analysis or determined changes over time can be used to determine patient health status and potentially predict or indicate worsening conditions. Moreover, changes over time can be used to evaluate specific posture-related metrics, such as right-side bias during the night for heart failure patients or night restlessness, which can provide additional context for patient health assessment. By implementing the systems and methods described herein, the functionality of ambulatory medical devices and medical device systems can be improved, enhancing patient monitoring capabilities, providing improved patient outcomes and more efficient use of healthcare and medical device system and sensor resources.

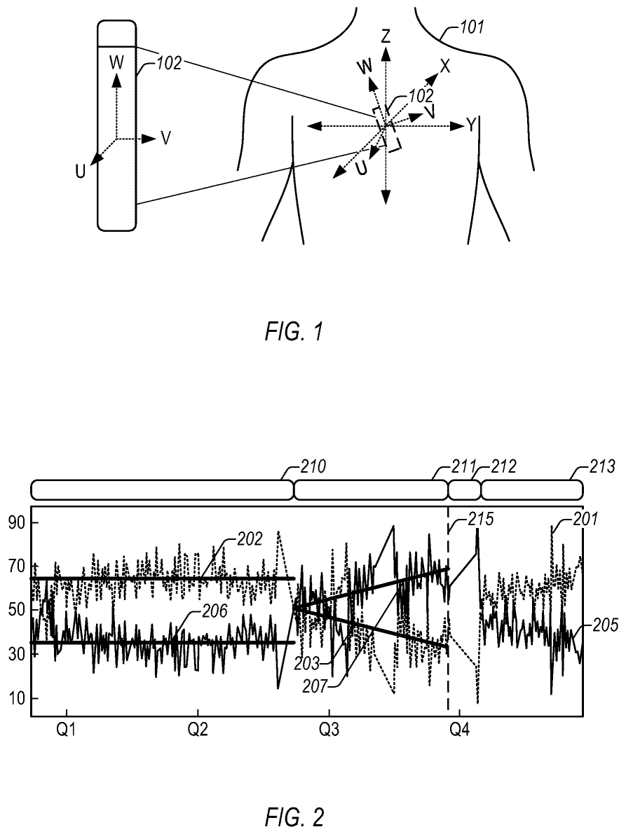

FIG. 1 illustrates an example ambulatory medical device 102, an insertable cardiac monitor (ICM), implanted in (or otherwise positioned on) a patient 101. The ambulatory medical device 102 can include a posture sensor, such as an accelerometer configured to sense acceleration information of the patient in local three-dimensional coordinates or one or more other posture sensors (e.g., a gyroscope, an inertial measurement unit, a magnetometer, a pressure sensor, a flex sensor, a strain sensor, a capacitive sensor, etc.), in this example a three-dimensional UVW space with respect to the ambulatory medical device 102 (or alternatively with the posture sensor in the ambulatory medical device 102). In contrast to the three-dimensional UVW space, separate patient three-dimensional coordinates are illustrated, in this example a three-dimensional XYZ space with respect to the patient 101. To accurately identify or determine posture information of the patient 101 using acceleration information of the patient 101, the UVW space of the ambulatory medical device 102, which can be at any orientation once implanted in or positioned on the patient 101, and in certain examples can migrate, rotate, or flip after implant or placement, must be translated into the XYZ space of the patient 101, or vice versa.

Patient posture patterns or trends can change with changes in patient health status. Changes in patient health status, including acute events, often follow or can be detected by specific changes in patient posture patterns or trends. Similarly, patient recovery from acute events can be detected as a recovery to patient posture patterns or trends from before the acute event or before a worsening patient status or condition that preceded the acute event.

FIG. 2 illustrates example patient posture trends determined using changes in patient upright posture information 201 and patient supine posture information 205, in the months (in quarters Q1, Q2, Q3, and Q4) preceding and following a heart failure event 215 through different periods, including a baseline period 210, a worsening period 211, a hospitalization period 212, and a recovery period 213.

In the baseline period 210, both of the patient upright posture information 201 and the patient supine posture information 205 illustrate substantially stable trends (e.g., changing less than a threshold percentage), a first upright trend 202 and a first supine trend 206 respectively. In the worsening period 211, the patient upright posture information 201 and the patient supine posture information 205 cross over and diverge, with a second upright trend 203 decreasing and a second supine trend 207 increasing until occurrence of the heart failure event 215 and the hospitalization period 212. In the recovery period 213, such as after discharge, the patient upright posture information 201 and the patient supine posture information 205 return towards their values from the baseline period 210.

In certain examples, a detected cross over of patient posture information, such as of patient upright posture information 201 and patient supine posture information 205, and in certain examples subsequent divergence, can be used to determine a patient health status (e.g., a patient health metric, etc.) indicative of a worsening patient health status or an increasing likelihood of an adverse acute event. In other examples, a subsequent cross over or returning trend towards baseline values can be used to determine a patient health status indicative of an improving patient health status or patient recovery.

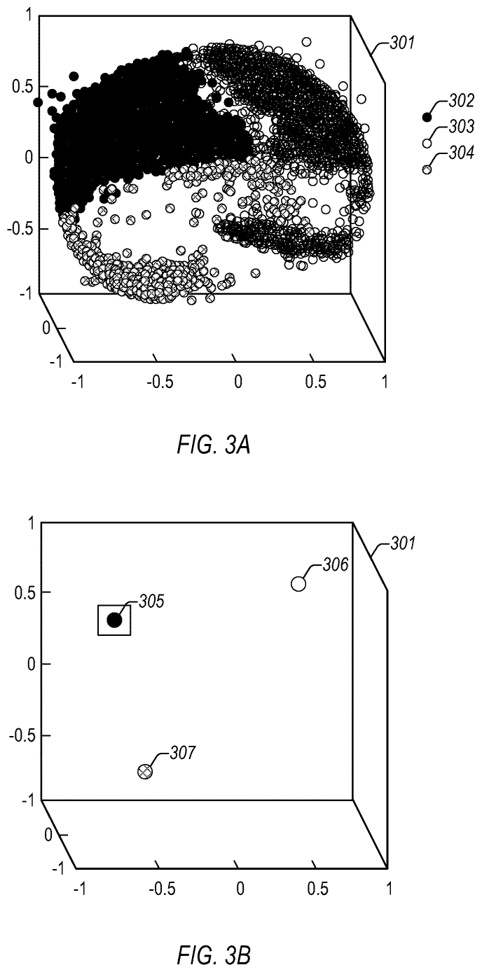

FIGS. 3A-3B illustrate initial first, second, and third clusters 302, 303, 304 of received acceleration information and determined respective first, second, and third centroids 305, 306, 307 of the initial clusters in UVW space 301 with respect to the accelerometer or the ambulatory medical device comprising the accelerometer. FIG. 3A illustrates acceleration information over a first time period. FIG. 3B illustrates identified centroids of the clusters in FIG. 3A.

In an example, the initial first, second, and third clusters 302, 303, 304 can include a supine/upright cluster, a first side cluster, and a second side cluster. In an example, the supine/upright cluster can be identified from the initial clusters as the initial cluster having the lowest opposite density measurement, as the opposite density measurements of the first and second side clusters will include the opposite sides, and based on the assumption that the patient is not likely to be spending time upside down. The first and second side clusters can be identified as having the highest opposite density measurement, and the first and second sides can be identified as the left and right sides based on their orientation with respect to the supine/upright information. In FIG. 3B, the box around the first centroid 305 indicates that such centroid has been identified as the supine/upright cluster in the manner described herein.

In certain examples, if more than three initial clusters are identified, such that the initial clusters can include separate supine and upright clusters, the upright cluster (as opposed to the supine/upright cluster) can be identified as the initial cluster having the lowest opposite density measurement. Like above, the first and second side clusters can be identified as having the highest opposite density measurement, and the first and second sides can be identified as the left and right sides based on their orientation with respect to the supine and upright information.

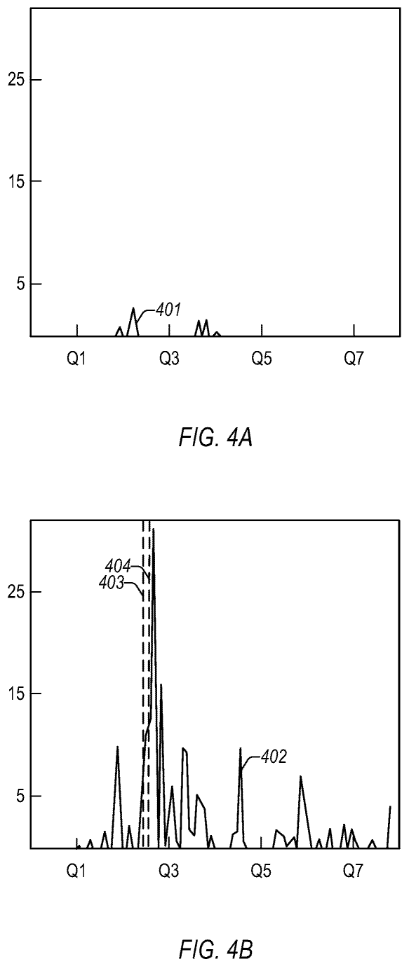

FIGS. 4A-4B illustrate example changes in posture distribution over time in control and heart failure patients, respectively. FIG. 4A illustrates standardized changes in posture distribution 401 over time (in quarters) for a control patient with no heart failure events and FIG. 4B illustrates standardized changes in posture distribution over time 402 for a heart failure patient with first and second heart failure events 403, 404.

To determine the patient posture trends, changes in the distribution of postures throughout the day can be measured and trended over time. For example, a percentage of data points in each posture for each day of data can be calculated. In certain examples, a heatmap of posture percentages over time can be created to provide a visualization of posture changes. Abnormal changes between consecutive days or time periods can be detected, where abnormal is relative with respect to a baseline or previous changes. In certain examples, a baseline can be created using an initial time period (e.g., 7 days, 14 days, etc.) of sensor measurements from the posture sensor. In other examples, the baseline can include other time periods, such as of longer time periods (e.g., one month, three months, etc.) or of known patient status.

In an example, abnormal changes can be determined using one or more methods, such as Jenson-Shannon Divergence or one or more other methods. Jensen-Shannon divergence involves calculating divergence between consecutive measurements, in this example, daily posture percentages, and in certain examples standardizing by taking each result with respect to the determined baseline. A detected change in posture patterns relative to the baseline can be indicative of changes in patient health. Based on certain assumptions, for example, that less activity, less upright time and more supine time, are indicative of worsening patient condition, certain detected changes can be indicative of a worsening patient health status, just as others (e.g., the opposite of those above) can be indicative of improving patient health status. Such methods provide a sensitive measure for monitoring posture trends and potentially identifying worsening patient health status or acute events based on significant deviations from typical posture distributions.

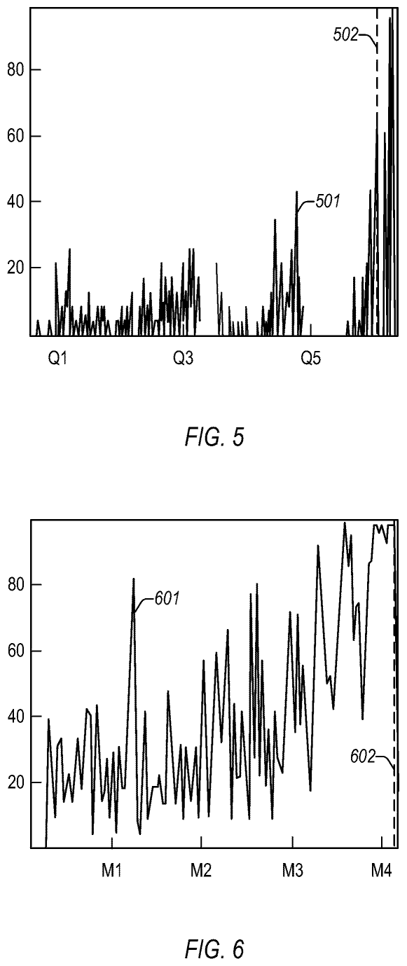

FIG. 5 illustrates an example change in night side bias 501 over time (in months) before a heart failure event 502. Right and left side posture percentages can be calculated each day for a patient during nighttime (e.g., from midnight to 8 am, using a sleep sensor, etc.). The night side bias 501 can be determined using a difference between right and left side posture percentages from a baseline. A change in night side bias can be indicative of physiologic changes to the patient and, additionally, can be a diagnostic indicator of a cause of heart failure for the patient. For example, there are separate indications for right and left-side heart failure, depending on the specific root cause of heart failure for the patient. Changes from a baseline can be indicative of worsening patient health status.

Another nighttime or sleep metric can include a combined sleep angle and left side avoidance metric to provide further insight into patient risk levels associated with posture patterns during sleep. Low, medium, and high-risk groups can be determined based on sleep posture characteristics and comparison to baseline measures or clinical information. For example, baseline measures can be compared to expected clinical values. If the baseline itself is abnormal, adherence thereto can be itself abnormal. In other examples, patients exhibiting a normal sleep angle and no left side avoidance can be classified as low risk, indicating typical sleep posture patterns. A medium-risk classification can be assigned to patients who demonstrate either an inclined sleep angle or left side avoidance, suggesting potential physiological changes or compensatory behaviors. A high-risk classification can be assigned to patients who display both an inclined sleep angle and left side avoidance, which may be indicative of more severe underlying conditions or advanced stages of heart failure. These risk classifications can be used in conjunction with other posture-based metrics to provide a more comprehensive assessment of patient health status and potentially predict or detect worsening conditions or acute events.

Additionally, different risk levels can be used to trigger sensor changes. For example, at low risk, posture can be determined at a first interval (e.g., every 20 minutes). At medium or high risk, posture can be determined at second or third shorter intervals (e.g., every 10 minutes for medium risk, every 2 minutes for high risk, etc.). Although discussed with respect to FIG. 5 as risk levels determined as composite classifications of sleep posture, in other examples, other changes in posture information, such as a determined change from a baseline amount greater than a threshold, or other physiologic information or combinations thereof, can trigger corresponding sensor changes.

FIG. 6 illustrates an example change in nighttime tossing percentage 601 over time (in months) before a heart failure event 602. In certain examples, posture detection can occur at specific intervals, such as every minute, 2 minutes, 5 minutes, 10 minutes, 20 minutes, etc., and a label for the detected posture can be created for each specific interval. In other examples, posture labels can be determined for longer specific time periods comprising multiple specific intervals using a local maximum of detected postures within that specific time period. For example, if posture is detected every minute, a posture label for a 20-minute or a one-hour time period can be determined as the posture having the greatest frequency in that time period. In an example, the nighttime tossing percentage 601 can be determined as a calculated percentage of consecutive intervals or time periods having different posture labels. An increase in nighttime tossing percentage 601 can be indicative of patient discomfort or worsening patient health status.

In other examples, posture data itself can be combined and viewed at once across a time period instead of as a comparison of multiple days. For example, metrics for each posture can be computed across a time period. Changes in successive time periods or separately trends or comparisons to a baseline can provide information about the patient health status. In an example, aggregate deviation from the baseline can be computed as a posture deviation score for one or multiple different postures. Heart failure and other adverse medical events are often preceded by a change in posture. Accordingly, a deviation above a specific threshold, or separately a trend or other rate of change (e.g., increase or decrease) continuing for a period of time (e.g., rising deviation above a threshold rate sustained over months) can trigger one or more alerts or transitions and otherwise be indicative of high risk, a worsening patient health status, or an indication of a future adverse medical event.

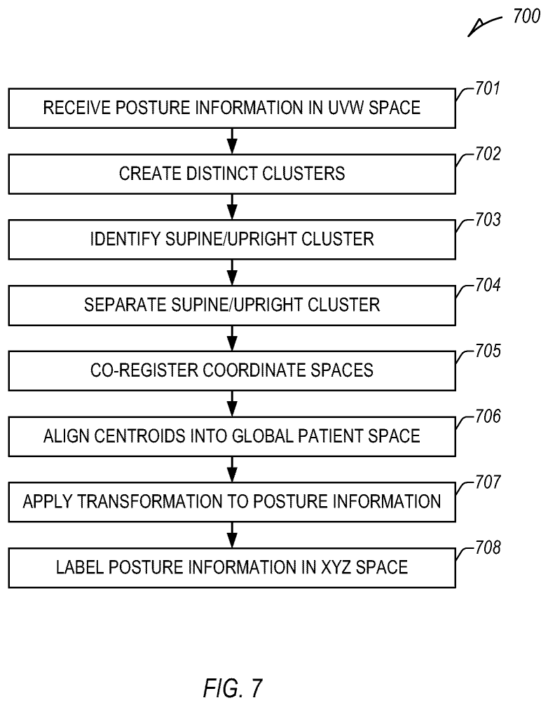

FIG. 7 illustrates an example method 700 to detect patient posture using posture information from a posture sensor of an ambulatory medical device. In an example, the posture information can include three-dimensional posture information with respect to the posture sensor of the ambulatory medical device. In certain examples, the posture sensor can include an accelerometer (e.g., a three-dimensional accelerometer) and the posture information can include acceleration information (e.g., three-dimensional acceleration information) of the patient.

At step 701, posture information (e.g., acceleration information, etc.) can be received, such as using a signal receiver circuit or one or more other circuits, etc. In an example, the posture information can include information from the posture sensor in multi-dimensional space, such as three-dimensional UVW space with respect to the posture sensor or the ambulatory medical device including the posture sensor or one or more other multi-dimensional spaces (e.g., two-dimensions, etc.).

At step 702, distinct clusters of the received posture information can be created in multi-dimensional space (e.g., three-dimensional space), such as using an assessment circuit or one or more other circuits, etc. For example, different clustering algorithms can be applied to three-dimensional data group points based on their spatial similarity or values to identify patterns and structures. Example algorithms can include K-Means, Hierarchical Clustering, DBSCAN, and Gaussian Mixture Models (GMM), each varying in their handling of cluster shape, density, and size.

In an example, at least two, two or more, or three or more different clusters can be initially identified, wherein at least one of the initial clusters includes a supine or upright cluster. The present inventors found, unexpectedly, that determination of posture starting with a large number of initial categories (e.g., equal to the final categories, or greater) performed unexpectedly worse than determination of posture starting with three or less initial categories, with one, two, or three categories performing significantly better than five or ten, etc. In an example, the supine or upright cluster can include a supine cluster, an upright cluster, or a combination supine/upright cluster. Another cluster can include a first side cluster, a second side cluster, or a combination first and second side cluster. In an example, the initial clusters can include a combination supine/upright cluster and separate first and second side clusters.

At step 703, the supine or upright cluster (e.g., the supine/upright cluster, etc.) can be identified, such as using the assessment circuit or one or more other circuits, etc. In an example, an opposite density measure can be determined for each of the initial clusters, indicative of a density of the posture information opposite the respective cluster in the multi-dimensional space with respect to the posture sensor (e.g., the UVW space, etc.), and the supine or upright cluster can be identified or determined as the respective cluster having the lowest opposite density measure.

In an example, each cluster can be defined as a convex hull, a convex shape (e.g., a smallest convex shape) that encloses a set of points in a given space, and the density measure can be determined as a spread of the points in the convex hull, or a measure of the number of points in the area of the convex shape. To determine the opposite density measure, an opposite cluster can be reflectively created that mirrors a respective cluster, and a density measure can be determined for that opposite cluster.

At step 704, the supine or upright cluster (e.g., the supine/upright cluster, etc.) can be separated, using the assessment circuit or one or more other circuits, etc., into a supine cluster and an upright cluster (separate from the supine cluster). For example, principal component analysis (PCA) can be performed on the information from the supine or upright cluster to separate the upright cluster from the supine cluster based on measurements (e.g., density, spread, etc.) of or across the cluster. In certain examples, following separation of the supine cluster and the upright cluster, the remaining clusters can be identified in relation thereto. In an example, the posture information for the patient (e.g., time spent in specific postures, transitions therebetween, trends, etc.) can be determined based on the identified clusters.

At step 705, the coordinate space of the device (e.g., the UVW space with respect to the posture sensor or the ambulatory medical device) and the patient (e.g., XYZ space with respect to the patient) can be co-registered (e.g., aligned or transformed), such as using an assessment circuit or one or more other circuits, etc. In an example, centroids of the clusters can be determined representative of each cluster and used for co-registration with ideal body coordinates of the patient. For example, a plane can be created using three centroids (e.g., supine, left, and right centroids, etc.) in the device space and rotated or transformed into a specific position in the patient space corresponding to the three centroids.

At step 706, centroids from the device space (e.g., the UVW space) can be aligned in the patient space (e.g., the XYZ space), such as using the assessment circuit or one or more other circuits, etc., based on the rotation from step 705. In certain examples, the aligned centroids can be compared to ideal patient locations in the patient space.

At step 707, the posture information from the device space (e.g., the UVW space) can be transformed, such as using the assessment circuit or one or more other circuits, etc., to the patient space (e.g., the XYZ space) based on the rotation from step 705 or the alignment from step 706.

At step 708, the posture information can be labeled in the patient space (e.g., the XYZ space), such as using the assessment circuit or one or more other circuits, etc., based on centroids of the identified clusters, and provided for output, such as for display by a user interface or as input to one or more other circuits or components, for example, to control or adjust a process or function of the ambulatory medical device or one or more other components of a medical device system including the ambulatory medical device or otherwise associated with the patient.

Although illustrated as a series of steps above, in certain examples, one or more steps are optional, and in other examples, different combinations or permutations of these or other steps or examples can be combined to form other methods or processes, which is also applicable to other examples discussed herein.

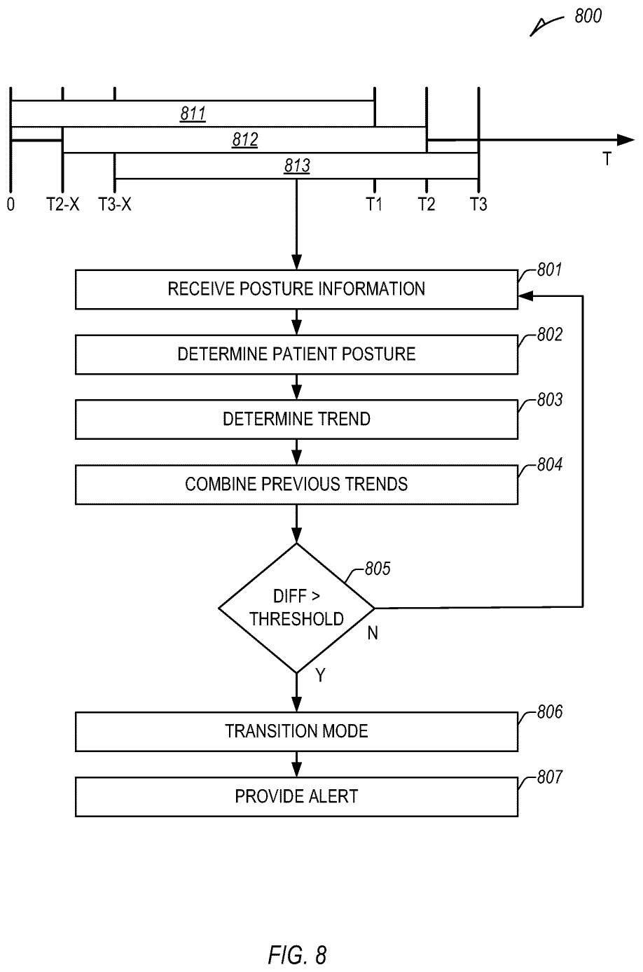

FIG. 8 illustrates an example method 800 to optimize medical device system resource allocation (e.g., improve resource allocation) using determined patient posture information, such as from one or more different overlapping or non-overlapping time periods (e.g., a first time period 811 (e.g., an initial time period) having a period of X, a second time period 812, a third time period 813, etc.). Repetition at specific time periods can be an effective self-or auto-calibration of posture determination.

At step 801, posture information (e.g., acceleration information, etc.) can be received with respect to one or more time periods, such as using a signal receiver circuit or one or more other circuits, etc. Although shown in step 801 as receiving information from the third time period 813, in certain examples the method 800 can include receiving posture information from multiple overlapping or non-overlapping time periods.

At step 802, patient posture can be determined, for example, with respect to a particular time period, such as using an assessment circuit or one or more other circuits, etc. In an example, posture determination can include one or more of the steps described in FIG. 7 or otherwise as described herein.

At step 803, one or more patient posture trends can be determined, such as using the assessment circuit or one or more other circuits, etc., based on the patient posture determined at step 802 in relation to a baseline or patient posture from one or more other time periods.

At step 804, the determined trend at step 803 can be combined with one or more previous trends, such as using the assessment circuit or one or more other circuits, etc.

At step 805, a difference between the determined trend from step 803 or the combined trends from step 804 can be compared to one or more thresholds. In an example, the threshold can include the combined trend, baseline information, or combinations thereof. For example, a difference between a value of the determined trend from step 803 (e.g., with respect to a specific time period) and a value of the combined trends from step 804 can be compared to one or more thresholds. In other examples, other values or differences can be determined (e.g., between trends for different or successive time periods, baseline information, etc.) and compared to the one or more thresholds. If the difference does not exceed the one or more thresholds, the method 800 can continue to the next time period (e.g., a fourth time period (not illustrated)). If the difference exceeds the one or more thresholds, the method 800 can proceed.

At step 806, one or more modes or processes or functions of the posture sensor, the ambulatory medical device, or one or more other components of a medical device system including the ambulatory medical device or otherwise associated with the patient can be transitioned, such as using the assessment circuit or one or more other circuits, etc.

At step 807, one or more alerts can be provided, such as using the assessment circuit or one or more other circuits, etc., based on the transition at step 806 or the comparison at step 805. In an example, an output can be provided of the determined patient posture at step 802, the determined trend at step 803, the combined trends at step 804, or the comparison at step 805 to a user interface for display to a user or to another circuit to control or adjust a process or a function of a medical device system including the ambulatory medical device or otherwise associated with the patient.

In certain examples, self-calibration can be invoked in one or more different ways, such as at implant (e.g., automatically, by clinician trigger, by patient trigger, etc.), periodically at one or more time periods (e.g., weekly, monthly, etc.), triggered by a user or another algorithm (e.g., by detected data drift or loss of signal from one or more other physiologic measures, upon detection of device movement, migration, or flip, etc.), or prompted by the method 800 itself, such as if a determined confidence of labeling is low, if a determined difference exceeds a threshold by a specific amount, etc.). For example, if a determined difference exceeds a threshold by a specific amount, a component of a medical device system, such as remote patient management device, etc., can provide an inquiry to the patient through one or more user interfaces (e.g., “In what position did you sleep last night: (A) fully supine; (B) supine with several pillows; (C) in a chair or semi-inclined; or (D) other?”, etc.).

Although illustrated as a series of steps above, in certain examples, one or more steps are optional, and in other examples, different combinations or permutations of these or other steps or examples can be combined to form other methods or processes, which is also applicable to other examples discussed herein.

### Medical Device System

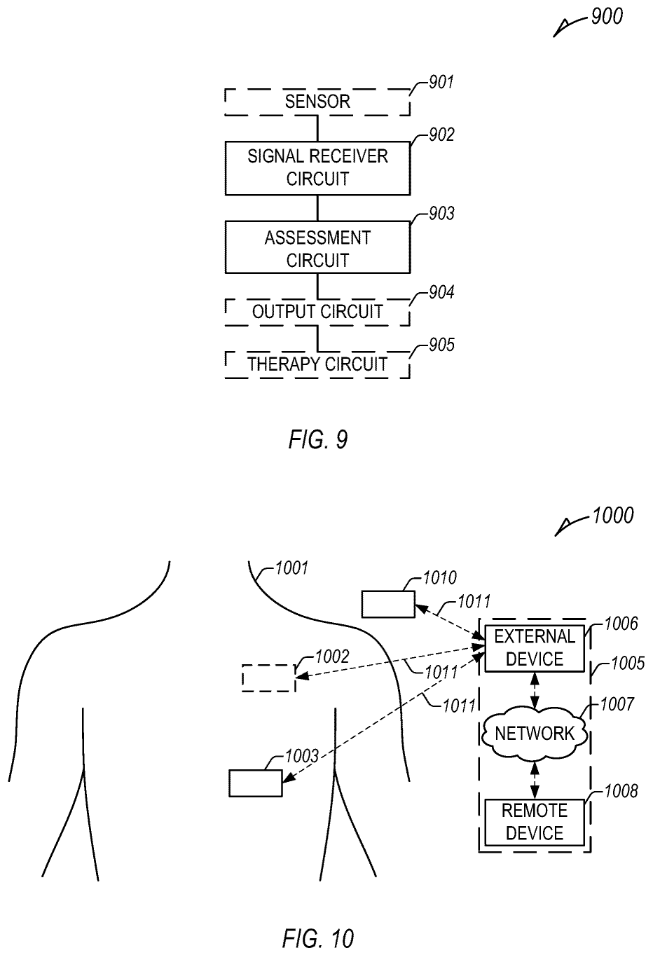

FIG. 9 illustrates an example system 900 (e.g., a medical device system). In an example, one or more aspects of the system 900 can be a component of, or communicatively coupled to, a medical device, such as an implantable medical device (IMD), an insertable cardiac monitor, an ambulatory medical device (AMD), etc. The system 900 can be configured to monitor, detect, or treat various physiologic conditions of the body, such as cardiac conditions associated with a reduced ability of a heart to sufficiently deliver blood to a body, including heart failure, arrhythmias, dyssynchrony, etc., or one or more other physiologic conditions and, in certain examples, can be configured to provide electrical stimulation or one or more other therapies or treatments to the patient.

The system 900 can include a single medical device or a plurality of medical devices implanted in a body of a patient or otherwise positioned on or about the patient to monitor patient physiologic information of the patient using information from one or more sensors, such as a sensor 901. In an example, the sensor 901 can include one or more of: a respiration sensor configured to receive respiration information (e.g., a respiratory rate, a respiration volume (tidal volume), etc.); an acceleration sensor (e.g., an accelerometer, a microphone, etc.) configured to receive cardiac acceleration information (e.g., cardiac vibration information, pressure waveform information, heart sound information, endocardial acceleration information, acceleration information, activity information, posture information, etc.); an impedance sensor (e.g., an intrathoracic impedance sensor, a transthoracic impedance sensor, a thoracic impedance sensor, etc.) configured to receive impedance information, a cardiac sensor configured to receive cardiac electrical information; an activity sensor configured to receive information about a physical motion (e.g., activity, steps, etc.); a posture sensor configured to receive posture or position information; a pressure sensor configured to receive pressure information; a plethysmograph sensor (e.g., a photoplethysmography sensor, etc.); a chemical sensor (e.g., an electrolyte sensor, a pH sensor, an anion gap sensor, a potassium sensor, a creatinine sensor, etc.); a temperature sensor; a skin elasticity sensor, or one or more other sensors configured to receive physiologic information of the patient.

The example system 900 can include a signal receiver circuit 902 and an assessment circuit 903. The signal receiver circuit 902 can be configured to receive physiologic information of a patient (or group of patients) from the sensor 901. The assessment circuit 903 can be configured to receive information from the signal receiver circuit 902, and to determine one or more parameters (e.g., physiologic parameters, stratifiers, etc.) or existing or changed patient conditions (e.g., indications of patient dehydration, respiratory condition, cardiac condition (e.g., heart failure, arrhythmia), sleep disordered breathing, etc.) using the received physiologic information, such as described herein. Physiologic information can include, among other things, one or more of: electrical information of the patient, such as cardiac electrical information (e.g., heart rate, heart rate variability, etc.), impedance information, temperature information, and in certain examples, respiration information (e.g., a respiratory rate, a respiration volume (tidal volume), etc.); mechanical information of the patient, such as cardiac acceleration information (e.g., cardiac vibration information, pressure waveform information, heart sound information, endocardial acceleration information, acceleration information, activity information, posture information, etc.), physical activity information (e.g., activity, steps, etc.), posture or position information, pressure information, plethysmograph information, and in certain examples, respiration information; chemical information; or other physiologic information of the patient. In an example, the signal receiver circuit 902 can include the sensor 901. In other examples, the signal receiver circuit can be coupled to or a component of the assessment circuit 903.

In certain examples, the assessment circuit 903 can aggregate information from multiple sensors or devices, detect various episodes using information from each sensor or device separately or in combination, update a detection status for one or more patients based on the information, and transmit a message or an alert to one or more remote devices that a detection for the one or more patients has been made or that information has been stored or transmitted, such that one or more additional processes or systems can use the stored or transmitted detection or information for one or more other review or processes.

In certain examples, such as to detect an improved or worsening patient condition, some initial assessment is often required to establish a baseline level or condition from one or more sensors or physiologic information. Subsequent detection of a deviation from the baseline level or condition can be used to determine the improved or worsening patient condition. However, in other examples, the amount of variation or change (e.g., relative or absolute change) in physiologic information over different time periods can used to determine a risk of an adverse medical event (such as a risk of a heart failure event, an arrhythmia episode, etc.), or to predict or stratify the risk of the patient experiencing an adverse medical event (e.g., a heart failure event, an arrhythmia episode, etc.) in a period following the detected change, in combination with or separate from any baseline level or condition.

Changes in different physiologic information can be aggregated and weighted based on one or more patient-specific stratifiers and, in certain examples, compared to one or more thresholds, for example, having a clinical sensitivity and specificity across a target population with respect to a specific condition (e.g., heart failure), etc., and one or more specific time periods, such as daily values, short term averages (e.g., daily values aggregated over a number of days), long term averages (e.g., daily values aggregated over a number of short term periods or a greater number of days (sometimes different (e.g., non-overlapping) days than used for the short term average)), etc.

The system 900 can include an output circuit 904 configured to provide an output to a user, or to cause an output to be provided to a user, such as through an output, a display, or one or more other user interface, the output including a score, a trend, an alert, or other indication. In other examples, the output circuit 904 can be configured to provide an output to another circuit, machine, or process, such as a therapy circuit 905 (e.g., a cardiac resynchronization therapy circuit, a chemical therapy circuit, a stimulation circuit, etc.), etc., to control, adjust, or cease a therapy of a medical device, a drug delivery system, etc., or otherwise alter one or more processes or functions of one or more other aspects of a medical device system, such as one or more cardiac resynchronization therapy parameters, drug delivery, dosage determinations or recommendations, etc. In an example, the therapy circuit 905 can include one or more of a stimulation control circuit, a cardiac stimulation circuit, a neural stimulation circuit, a dosage determination or control circuit, etc. In other examples, the therapy circuit 905 can be controlled by the assessment circuit 903, or one or more other circuits, etc. In certain examples, the assessment circuit 903 can include the output circuit 904 or can be configured to determine the output to be provided by the output circuit 904, while the output circuit 904 can provide the signals that cause the user interface to provide the output to the user based on the output determined by the assessment circuit 903.

### Efficiency and Mode Transitions

Ambulatory medical devices powered by rechargeable or non-rechargeable batteries, responsible for sensing physiologic signals and physiologic information of the patient, and in certain examples making determinations using such information, have to make certain tradeoffs between device battery life, or in the instance of implantable medical devices with non-rechargeable batteries, between device replacement periods often including surgical procedures, and device sensing, storage, processing, and communication characteristics, such as sensing resolution, sampling frequency, sampling periods, the number of active sensors, the amount of stored information, processing characteristics, or communication of physiologic information outside of the device.

Medical devices can include higher-power modes and lower-power modes. In certain examples, the low-power mode can include a low resource mode, characterized as requiring less power, processing time, memory, or communication time or bandwidth (e.g., transferring less data, etc.) than a corresponding high-power mode. The high-power mode can include a relatively higher resource mode (e.g., higher in comparison to the low-power mode), characterized as requiring more power, processing time, memory, or communication time or bandwidth than the corresponding low-power mode.

A technological problem in the art with respect to such devices exists that not all information can be stored, not all sensors can be active in a high-power or high-resolution mode, not all algorithms can be active, and not all sensed or processed information can be communicated outside of the device at all times without detrimentally impacting the lifespan of the devices. Technological solutions to such problems are often improvements in physical sensors, or alternatively in sensing and processing physiologic information in a way that improves device efficiency, extending the lifespan of the device, or to perform new determinations using existing sensors or information in a way that was not previously known, increasing the capabilities of an existing device without adding additional hardware to the device, or requiring additional sensors or hardware to be implanted in the patient. Efficiency improvements in one area can enable additional operation in another, improving the technical capabilities of existing devices having real-world constraints.

For example, physiologic information, such as indicative of a potential adverse physiologic event, can be used to transition from a low-power mode to a high-power mode. However, by the time physiologic information detected in the low-power mode indicates a possible event, valuable information has been lost, unable to be recorded in the high-power mode.

Another technological problem exists in that false or inaccurate determinations that trigger a high-power mode unnecessarily unduly limit the usable life of certain ambulatory medical devices. For numerous reasons, it is advantageous to accurately detect and determine physiologic events, and to avoid unnecessary transitions from the low-power mode to the high-power mode to improve use of medical device resources.

In an example, a change in modes can enable higher resolution sampling or an increase in the sampling frequency or number or types of sensors used to sense physiologic information leading up to and including a potential event. Different physiologic information is often sensed using non-overlapping time periods of the same sensor, in certain examples, at different sampling frequencies and power costs.

In certain examples, a high-power mode can be in contrast to a low-power mode, and can include one or more of: enabling one or more additional sensors, transitioning from a low-power sensor or set of sensors to a higher-power sensor or set of sensors, triggering additional sensing from one or more additional sensors or medical devices, increasing a sensing frequency or a sensing or storage resolution, increasing an amount of data to be collected, communicated (e.g., from a first medical device to a second medical device, etc.), or stored, triggering storage of currently available information from a loop recorder in long-term storage or increasing the storage capacity or time period of a loop recorder, or otherwise altering device behavior to capture additional or higher-resolution physiologic information or perform more processing, etc.

Additionally, or alternatively, event storage can be triggered. Information sensed or recorded in the high-power mode can be transitioned from short-term storage, such as in a loop recorder, to long-term or non-volatile memory, or in certain examples, prepared for communication to an external device separate from the medical device. In an example, cardiac electrical or cardiac mechanical information leading up to and in certain examples including the detected event (e.g., a heart failure event, an arrhythmia episode, etc.) can be stored, such as to increase the specificity of detection. In an example, multiple loop recorder windows (e.g., 2-minute windows) can be stored sequentially. In systems without early detection, to record this information, a loop recorder with a longer time period would be required at substantial additional cost (e.g., power, processing resources, component cost, etc.).

For example, ambulatory medical devices frequently contain one or more accelerometer sensors and corresponding processing circuits to determine and monitor patient acceleration information, such as, among other things, cardiac vibration information associated with blood flow or movement in the heart or patient vasculature (e.g., heart sounds, cardiac wall motion, etc.), patient physical activity or position information (e.g., patient posture, activity, etc.), respiration information (e.g., respiratory rate (RR), tidal volume (TV), rapid shallow breathing index (RSBI), respiration phase, breathing sounds, etc.), etc. In one example, heart sounds and patient activity can be detected using non-overlapping time periods of the same, single-or multi-axis accelerometer, at different sampling frequencies and power costs.

In an example, a transition to a high-power mode can include using the accelerometer to detect heart sounds throughout the high-power mode, or at a larger percentage of the high-power mode than during a corresponding low-power mode, etc. In other examples, waveforms for medical events can be recorded, stored in long-term memory, and transferred to a remote device for clinician review. In certain examples, only a notification that an event has been stored is transferred, or summary information about the event. In response, the full event can be requested for subsequent transmission and review. However, even in the situation where the event is stored and not transmitted, resources for storing and processing the event are still by the medical device.

Another technological problem exists in that suboptimal programming of device parameters and parameter settings can negatively impact functionality of ambulatory medical devices. Accordingly, identifying suboptimal programming by clinicians and other caregivers and generating and providing alerts or notifications of such identified suboptimal programming, or reprogramming recommendations, and in certain examples, reprogramming ambulatory medical devices directly, can improve the functionality of existing ambulatory medical devices without requiring other improvements to the hardware of devices providing therapy or the sensors themselves.

### Implant and Follow-up

When receiving a new medical device, patients may need to try several sets of parameter settings to receive sufficient or optimal therapy. In addition, in-clinic follow-up appointments are currently required as patient condition can change or response to existing devices or therapy can change over time, requiring additional changes to parameter settings that at one time were sufficient or optimal. In typical operation, a medical device, such as a cardiac resynchronization therapy device, is first programmed at a time of implant to a first operating mode, such as with respect to a first set of parameter settings, and then adjusted during scheduled in-clinic follow-up procedures with a clinician. Initial follow-up after implant (e.g., post-implant) or programming changes is generally a first time period, such as 4-6 weeks to allow patient recovery from the implant procedure and determination of baseline measurements for the patient from which to base future operation and monitoring, as well as to compare patient condition after implant or programming changes to the patient condition pre-implant or previous to programming changes. Subsequent follow-up after the initial follow-up can be less frequent, occurring, for example, every 3-6 months (e.g., at 6 months post-implant, at 12-months post-implant, etc.), or more or less as needed (e.g., between 1 and 12 months, etc.) depending on programming changes or changes in patient health status or condition (e.g., a patient response metric). However, traditional follow-up appointments are in-person in a clinical setting and require travel for the patient, often at a substantial burden. In addition, programming changes may require additional follow-up, such as a new initial follow-up appointment and observation time, substantially increasing resources associated with programming the medical device and reducing the usable lifespan of the medical device during which the device is using limited resources to provide sufficient or optimal therapy.

In other examples, follow-up schedules can be determined or prioritized based on information from the ambulatory medical device, and moreover, changes can be made remotely, without in-person follow-up appointments, increasing the usable lifespan of the limited resources of the ambulatory medical device.

For implantable medical devices, once implanted and in certain examples after a recovery period, the implantable medical device can begin in a first mode, such as a cardiac resynchronization therapy mode, a monitoring mode, or one or more other therapy modes having different parameter settings, depending on the patient or clinician. In certain examples, a stimulation circuit can generate and provide one or more stimulation signals in one or more stimulation modes, and the assessment circuit can be configured to control the stimulation circuit, such as to adjust one or more parameters or transition between different therapy modes, etc.

### Physiologic Parameters

In certain examples, physiologic information of a patient can be sensed using one or more sensors located within, on, or proximate to the patient, such as a cardiac sensor, a heart sound sensor, or one or more other sensors described herein. For example, cardiac electrical information of the patient can be sensed using a cardiac sensor. In other examples, cardiac acceleration information of the patient can be sensed using a heart sound sensor. The cardiac sensor and the heart sound sensor can be components of one or more (e.g., the same or different) medical devices (e.g., an implantable medical device, an ambulatory medical device, etc.). Timing metrics between different features (e.g., first and second cardiac features, etc.) can be determined, such as by a processing circuit of the cardiac sensor or one or more other medical devices or medical device components, etc. In certain examples, the timing metric can include an interval or metric between first and second cardiac features of a first cardiac interval of the patient (e.g., a duration of a cardiac cycle or interval, a QRS width, etc.) or between first and second cardiac features of respective successive first and second cardiac intervals of the patient. In an example, the first and second cardiac features include equivalent detected features in successive first and second cardiac intervals, such as successive R waves (e.g., an R-R interval, etc.) or one or more other features of the cardiac electrical signal, etc.

Heart sounds are recurring mechanical signals associated with cardiac vibrations or accelerations from blood flow through the heart or other cardiac movements with each cardiac cycle or interval and can be separated and classified according to activity associated with such vibrations, accelerations, movements, pressure waves, or blood flow. Heart sounds include four major features: the first through the fourth heart sounds (S1 through S4, respectively). The first heart sound (S1) is the vibrational sound made by the heart during closure of the atrioventricular (AV) valves, the mitral valve and the tricuspid valve, and the opening of the aortic valve at the beginning of systole, or ventricular contraction. The second heart sound (S2) is the vibrational sound made by the heart during closure of the aortic and pulmonary valves at the beginning of diastole, or ventricular relaxation. The third and fourth heart sounds (S3, S4) are related to filling pressures of the left ventricle (LV) during diastole. An abrupt halt of early diastolic filling can cause the third heart sound (S3). Vibrations due to atrial kick can cause the fourth heart sound (S4). Valve closures and blood movement and pressure changes in the heart can cause accelerations, vibrations, or movement of the cardiac walls that can be detected using an accelerometer or a microphone, providing an output referred to herein as cardiac acceleration information.

In an example, heart sound signal portions, or values of respective heart sound signals for a cardiac interval, can be detected as amplitudes occurring with respect to one or more cardiac electrical features or one or more energy values with respect to a window of the heart sound signal, often determined with respect to one or more cardiac electrical features. For example, the value and timing of an S1 signal can be detected using an amplitude or energy of the heart sound signal occurring at or about the R wave of the cardiac interval, and the value and timing of an S2 signal can be detected using an amplitude or energy of the heart sound signal occurring at or about the closure of the aortic and pulmonary valves, marking the transition from systole to diastole. S3 and S4 signal portions can be determined, such as by a processing circuit of the heart sound sensor or one or more other medical devices or medical device components, etc. In certain examples, the S3 signal portion can include a value or energy of the heart sound signal occurring in an S3 window of the cardiac interval, and the S4 signal portion can include a value or energy of the heart sound signal occurring in an S4 window of the cardiac interval. The S3 window occurs after S2 (e.g., 100 ms-200 ms after S2, 150 ms-200 ms after S2, etc.) and lasts for an S3 interval (e.g., 100 ms, 200 ms, etc.). The S4 window occurs shortly before the R wave or S1 (ending before or at the R wave or S1) and lasts for an S4 interval (e.g., 50 ms, 100 ms, 200 ms, etc.). The S3 or S4 windows and intervals can be determined as a set time period in the cardiac interval with respect to one or more other cardiac electrical or mechanical features, such as forward from one or more of the R wave, the T wave, or one or more features of a heart sound waveform or backwards from a subsequent R wave or a detected S1 of a subsequent cardiac interval. In certain examples, the length of the S3 or S4 intervals can depend on one or more factors, such as heart rate or patient characteristics, etc.

In an example, a heart sound parameter can include information of or about multiple of the same heart sound parameter or different combinations of heart sound parameters over one or more cardiac cycles or a specified time period (e.g., 1 minute, 1 hour, 1 day, 1 week, etc.). For example, a heart sound parameter can include a composite first heart sound (S1) parameter representative of a plurality of S1 parameters, for example, over a certain time period (e.g., a number of cardiac cycles, a representative time period, etc.), or one or more other heart sounds (e.g., a second heart sound (S2), a third heart sound (S3), a fourth heart sound (S4), etc.), etc.

In an example, the heart sound parameter can include an ensemble average of a particular heart sound over a heart sound waveform, such as that disclosed in the commonly assigned Siejko et al. U.S. Pat. No. 7,115,096 entitled “THIRD HEART SOUND ACTIVITY INDEX FOR HEART FAILURE MONITORING,” or in the commonly assigned Patangay et al. U.S. Pat. No. 7,853,327 entitled “HEART SOUND TRACKING SYSTEM AND METHOD,” each of which are hereby incorporated by reference in their entireties, including their disclosures of ensemble averaging an acoustic signal and determining a particular heart sound of a heart sound waveform. In other examples, the signal receiver circuit can receive the at least one heart sound parameter or composite parameter, such as from a heart sound sensor or a heart sound sensor circuit.

In an example, cardiac electrical information of the patient can be received, such as using a signal receiver circuit of a medical device, from a cardiac sensor (e.g., one or more electrodes, etc.) or cardiac sensor circuit (e.g., including one or more amplifier or filter circuits, etc.). In an example, the received cardiac electrical information can include the timing metric between the first and second cardiac features of the patient. In an example, cardiac acceleration information of the patient can be received, such as using the same or different signal receiver circuit of the medical device, from a heart sound sensor (e.g., an accelerometer, etc.) or heart sound sensor circuit (e.g., including one or more amplifier or filter circuits, etc.). In certain examples, additional physiologic information can be received, such as one or more of heart rate information, activity information of the patient, or posture information of the patient, from one or more other sensor or sensor circuits.

Respiration information can include, among other things, a respiratory rate (RR) of the patient, a tidal volume (TV) of the patient, a rapid shallow breathing index (RSBI) of the patient, or other respiratory information of the patient. The respiratory rate is a measure of a breathing rate of the patient, generally measured in breaths per minute. The tidal volume is an aggregate measure of respiration changes, such as detected using measured changes in thoracic impedance, etc. The RSBI is a measure (e.g., a ratio) of respiratory frequency relative to (e.g., divided by) tidal volume of the patient. The nHR is a measure of heart rate (HR) of the patient at night, either in relation to sensing patient sleep or using a preset or selectable time of day corresponding to patient sleep. In certain examples, respiration information of the patient can be determined using changes in impedance information and accordingly can be considered electrical information, but different than cardiac electrical information. In other examples, respiration information of the patient can be determined using changes in activity or acceleration information and accordingly can be considered mechanical information.

Physiologic metrics, as described herein, or measures or indications of physiologic information, can include one or more different measures of rate, amplitude, energy, etc., of different physiologic information over one or more time periods, such as representative daily values, etc. For example, heart sound metrics can be determined for each heart sound (e.g., the first heart sound (S1) through the fourth heart sound (S4), etc.) and can include an indication of an amplitude or energy of a specific heart sound for a specific cardiac cycle, or a representation of a number of cardiac cycles of the patient over a specific time period. Daily metrics can be determined representative of an average daily value for the patient, either corresponding to a waking time or a 24-hour period, etc. Respiration metrics can include, among other things, a mean or median respiratory rate, binned values of rates, and a representative value of specific rate bins, etc. Heart rate metrics can include an average nighttime heart rate, a minimum nighttime heart rate, heart rate at rest, etc.

The activity information can include an activity measurement of the patient, such as detected using an accelerometer, a posture sensor, a step counter, or one or more other activity sensors associated with an ambulatory medical device. Activity may be used to gate other physiologic measurements such as heart rate or respiratory rate so that the change in these metrics with increased patient activity may be used to infer patient cardiovascular and metabolic status including measurement of oxygen consumption. The impedance information can include, among other things, thoracic impedance information of the patient, such as a measure of impedance across a thorax of the patient from one or more electrodes associated with the ambulatory medical device (e.g., one or more leads of an implantable medical device proximate a heart of the patient and a housing of the implantable medical device implanted subcutaneously at a thoracic location of the patient, one or more external leads on a body of the patient, etc.). In other examples, the impedance information can include one or more other impedance measurements associated with the thorax of the patient, or otherwise indicative of patient thoracic impedance.

The temperature information can include an internal patient temperature at an ambulatory medical device, such as implanted in the thorax of the patient, or one or more other temperature measurements made at a specific location on the patient, etc. The temperature information can be detected using a temperature sensor, such as one or more circuits or electronic components having an electrical characteristic that changes with temperature. The temperature sensor can include a sensing element located on, at, or within the ambulatory medical device configured to determine a temperature indicative of patient temperature at the location of the ambulatory medical device.

In contrast to and separate from the electrical or mechanical information discussed above, the chemical information can include information about one or more chemical properties of blood, interstitial space (e.g., the space between cells, such as including interstitial fluid), or other tissue (e.g., muscle tissue, fat tissue, organ tissue, etc.) of the patient, such as information indicative of or including one or more of a glucose level, pH level, dissolved gas level (e.g. oxygen, carbon dioxide, carbon monoxide, etc.), electrolyte level (e.g., sodium, potassium, calcium, etc.), organic compound level (e.g., lactate, cholesterol, hemoglobin, creatinine, etc.), or biologic compound level (e.g., enzymes, antibodies, receptors, etc.), etc. The chemical information may be measured by one or more of an electrical sensor, mechanical sensor, electrochemical sensor, biosensor (e.g., enzyme biosensor, etc.), ion-selective electrode sensor, optical sensor, etc. In an example, the chemical information may include potassium information (e.g., one or more of interstitial potassium information, serum potassium information, etc.), creatinine information (e.g., one or more of interstitial creatinine information, serum creatinine information, etc.), or combinations thereof.

In certain examples, interstitial chemical information, such as one or more chemical levels in an interstitial space (e.g., a space between one or more of connective tissue, muscle fibers, nervous tissue, etc.) or of interstitial fluid, etc., can be indicative of serum chemical information. For example, potassium may move between cells or tissue and interstitial fluid (e.g., a change in interstitial potassium level may be followed by or reflective of a change in serum potassium level or vice versa), such that chemical information on serum potassium can include interstitial potassium. In certain examples, one of interstitial or serum chemical information can lead or lag the other, such that a change in one can indicate a worsening patient condition is detectable before the other. In one example, interstitial potassium information can lead serum potassium information as an indicator of electrolyte imbalance.

### Alert States

In certain examples, an alert state (e.g., an in-alert state, an out-of-alert state, a priority alert state, etc.) of the patient can be adjusted or determined using information of the patient, such as to increase a sensitivity or specificity of alert state determination, reduce false positive alert state determinations, alert state transitions or adjustments, or otherwise reduce storage or transmission of physiologic information associated or transitions associated with false positive alert state determinations, and power and processing resources associated with the same. In an example, the alert state can be determined using a comparison of a value of the health index (e.g., a numerical value, etc.) to one or more fixed or adaptable alert thresholds (e.g., based at least in part on one or more relative factors, such as measurements from the patient over the past 30 days, etc.). In an example, the alert state can be provided to a user interface for display to a user or to a control circuit to control or adjust a process or function of the system. In an example, the alert state can include one or more of an indication, recommendation, or instruction to perform one or more actions (e.g., administer or provide a drug or class of drug, adjust or optimize a guideline-directed medical therapy (GDMT), etc.). For, example, a GDMT may advise administration of a quantity of a drug or a rate of increase in a dosage, etc. In an example, determination of an in-alert or priority alert state can trigger an indication or instruction to administer or provide a specific class of diuretic or to deviate from GDMT (e.g., increase GDMT above a standard recommendation, hold GDMT at a standard recommendation, hold GDMT at a current level, decrease GDMT below a standard recommendation, increase a dosage or rate of increase of a drug, reduce a dosage or rate of decrease of a drug, etc.).

In certain examples, the techniques described above or herein can be used in various combinations or permutations. For example, combinations or permutations of techniques described above or herein can be selected based upon patient history, patient treatment (e.g., in-patient care, out-patient care, etc.), clinician input, etc.

As used herein, high and low (or high, medium, and low, etc.) can be relative or categorical terms, in certain examples with respect to clinical or population values, patient-specific values (e.g., a representative value, such as a current value, with respect to a short-or long-term range of values, etc.), or combinations thereof. For example, a high value can include a value in an upper percentage (e.g., at or above an upper quartile, etc.) of values experienced by the patient over respective time periods, such as one or more of a short-term range (e.g., having a period between 1 week and 3 months, such as 1 month, etc.), a long term range (e.g., having a period greater than the short-term range, such as greater than 1 month, greater than 3 months, the last 6 months, or longer, etc.). A low value can include a value in a lower percentage (e.g., at or below a mean or median, below the upper quartile, etc.). A medium value can, in certain examples, include a value between the upper and lower quartiles or within a threshold percentage of a mean or median, etc. In other examples, values can be determined with respect to clinical or population values, in certain examples, further respective to matching patient demographics (e.g., age, sex, comorbidities, etc.) or type of medical device (e.g., CRT-D device, ICD device, etc.), etc.

In an example, determinations described herein can be used to change device behavior, trigger additional sensing, data processing, storage, or transmission, or otherwise alter one or more modes, processes, or functions of medical devices associated with such determinations. For example, determinations can require data over a substantial time period (e.g., multiple days, weeks, a month or more, etc.). Such determinations can be initially determined by the device at yearly or semi-yearly (e.g., every 6 months, every 3 months, etc.) by default, or triggered by worsening patient health status or upon instruction from a clinician or caregiver, etc. In a first example, an assessment circuit can determine one or more indications quarterly, consuming a default amount of device resources. If the quarterly determination exceeds one or more of a patient-specific or population threshold, the assessment circuit can alter device functionality to increase the frequency of making such determinations, increasing the use of device resources, in certain examples reducing device lifespan, but providing additional monitoring and determinations. In other examples, if a determination exceeds one or more thresholds, additional sensing can be triggered, such as enabling additional sensors, or sensing enabled sensors with a higher resolution or sampling frequency, storing more information, and communicating more information outside of the device, such as to an external programmer, or increasing the frequency of communication outside of the device, increasing the use of device resources, in certain examples reducing device lifespan, but providing additional monitoring and determinations.

In certain examples, determinations described herein can include one or more determined risk curves illustrating determined risks at different time periods into the future, such as a determined risk of mortality (e.g., cardiovascular death), a determined risk of heart failure hospitalization, etc. Information about the determined risks or the determined risk curves or portions of the determined risk curves themselves can be provided to a user, such as to a patient, clinician, caregiver, etc., or can be used to make one or more device changes, such as described herein (e.g., therapies, treatments, device settings, etc.), or trigger one or more other processes or notifications, etc.

### Patient Indications

Indications of patient condition (e.g., improved or worsening patient condition, etc.) can include single-feature determinations based on a single feature or measure of a single type of physiologic information, or separately a composite determination based on a combination of physiologic information, such as two or more separate features of physiologic measures. In addition, indications of patient condition can be device-based, such as determined using physiologic information detected from the patient using the one or more ambulatory medical devices without input of clinical information about the patient separate from that detected or sensed physiologic information. In other examples, indications of patient condition can be a combination of device-based and clinical-based information of the patient, such as clinician diagnosis or determination of risk, patient history, patient age, comorbidities, prior hospitalization, type of implanted device, etc. In certain examples, separate determinations can be made for different combinations of clinical information.

One example of a composite indication is the HeartLogic™ index, a HeartLogic™ in-alert time, or one or more other composite measurements or measures thereof. The HeartLogic™ index is a composite indication of patient condition determined using different combinations or weightings of physiologic information, including two or more of S1 heart sounds, S3 heart sounds, thoracic impedance, activity information, respiration information, and nighttime heart rate (nHR). The HeartLogic™ index can be indicative of a heart failure status, a risk a heart failure event (e.g., within in a given time period), or a worsening of the heart failure status or risk of heart failure event in the patient over time. The HeartLogic™ in-alert time is a measure of time that the HeartLogic™ index is above an alert threshold.

In certain examples, the different combinations or weightings of physiologic information used to determine the HeartLogic™ index can be adjusted or determined based on a risk stratifier. In certain examples, the risk stratifier can be determined as a different combination of physiologic information, including one or more of S3, respiratory rate, and time active (e.g., an amount of time at a specific activity level above a mean activity level of the patient or a specific threshold, etc.). For example, if the risk stratifier is low, or below a first threshold, the HeartLogic™ index can be determined using a first combination of physiologic information. If the risk stratifier is high, or above a second threshold, the HeartLogic™ index can be determined using a second combination of physiologic information, such as additional information than included in the first combination (e.g., the first combination and the second combination, etc.). If the risk stratifier is between the first and second thresholds, the HeartLogic™ index can be determined using the first combination and one or more metrics or components of the second combination, or using the first combination and the second combination, but with the second combination having less weight than if the risk stratifier is above the second threshold (e.g., using less of the second combination than the first combination).

In an example, the HeartLogic™ index and in-alert time can include worsening heart failure or physiologic event detection, including risk indication or stratification, such as that disclosed in the commonly assigned An et al. U.S. Pat. No. 9,968,266 entitled “RISK STRATIFICATION BASED HEART FAILURE DETECTION ALGORITHM,” or in the commonly assigned An et al. U.S. Pat. No. 9,622,664 entitled “METHODS AND APPARATUS FOR DETECTING HEART FAILURE DECOMPENSATION EVENT AND STRATIFYING THE RISK OF THE SAME,”or in the commonly assigned Thakur et al. U.S. Pat. No. 10,660,577 entitled “SYSTEMS AND METHODS FOR DETECTING WORSENING HEART FAILURE,” or in the commonly assigned An et al. U.S. Patent Application No. 2014/0031643 entitled “HEART FAILURE PATIENT STRATIFICATION,” or in the commonly assigned Thakur et al. U.S. Pat. No. 10,085,696 entitled “DETECTION OF WORSENING HEART FAILURE EVENTS USING HEART SOUNDS,” each of which are hereby incorporated by reference in their entireties, including their disclosures of heart failure and worsening heart failure detection, heart failure risk indication detection, and stratification of the same, etc.

### Patient Management System

FIG. 10 illustrates an example patient management system 1000 and portions of an environment in which the patient management system 1000 may operate. The patient management system 1000 can perform a range of activities, including remote patient monitoring and diagnosis of a disease condition, programming of ambulatory medical devices, and control of one or more therapies. Such activities can be performed proximal to a patient 1001, such as in a patient home or office, through a centralized server, such as in a hospital, clinic, or physician office, or through a remote workstation, such as a secure wireless mobile computing device.

The patient management system 1000 can include one or more medical devices, an external system 1005, and a communication link 1011 providing for communication between the one or more ambulatory medical devices and the external system 1005. The one or more medical devices can include an ambulatory medical device, such as an implantable medical device 1002, a wearable medical device 1003, or one or more other implantable, leadless, subcutaneous, external, wearable, or medical devices configured to monitor, sense, or detect information from, determine physiologic information about, or provide one or more therapies to treat various conditions of the patient 1001, such as one or more cardiac or non-cardiac conditions (e.g., dehydration, sleep disordered breathing, etc.).

In an example, the implantable medical device 1002 can include one or more cardiac rhythm management (CRM) devices implanted in a chest of a patient, having a lead system including one or more transvenous, subcutaneous, or non-invasive leads or catheters to position one or more electrodes or other sensors (e.g., a heart sound sensor) in, on, or about a heart or one or more other position in a thorax, abdomen, or neck of the patient 1001. In another example, the implantable medical device 1002 can include a monitor implanted, for example, subcutaneously in the chest of patient 1001, the implantable medical device 1002 including a housing containing circuitry and, in certain examples, one or more sensors, such as a temperature sensor, etc.

Cardiac rhythm management devices are generally configured to receive cardiac electrical information from, and in certain examples, provide electrical stimulation to, one or more electrodes located within, on, or proximate to the heart, such as coupled to one or more leads and located in one or more chambers of the heart, within the vasculature of the heart near one or more chambers, or otherwise attached to or in contact with or proximate to the heart. Cardiac rhythm management devices can include, among others, pacemakers, implantable cardioverter defibrillators (ICD), subcutaneous implantable cardioverter defibrillators, cardiac resynchronization therapy defibrillators (CRT-Ds), insertable cardiac monitors, leadless cardiac pacemakers (LCPs), or wearable or remote monitoring systems.

Cardiac resynchronization therapy (CRT) refers generally to stimulation therapy generated and provided to one or more chambers of the heart (e.g., frequently two or more of the right ventricle (RV), the left ventricle (e.g., commonly through the cardiac vasculature), or the right atrium (RA), etc.) to improve cardiac function, such as to improve coordination of contractions between different chambers of the heart (e.g., the right ventricle and the left ventricle, the right atrium and the right ventricle, etc.) or to otherwise improve cardiac output or efficiency. Cardiac resynchronization therapy can include biventricular pacing (e.g., both right and left ventricular pacing), single-chamber pacing (e.g., right ventricle pacing, left ventricle pacing, etc.), sensing or pacing in one or more other chambers or combinations of chambers (e.g., right atria, etc.), as well as multi-site pacing (MSP) (e.g., applying one or more stimulation signals to multiple (e.g., two or more) electrodes in or proximate to a chamber (e.g., commonly the left ventricle, but also in certain examples the right ventricle, the right atrium, or combinations thereof) for a single cardiac cycle), and in certain examples, HIS-bundle pacing, septal pacing, etc. The timing of stimulation signals in the cardiac cycle or with respect to one or more cardiac events often varies depending on a number of factors, including placement of the lead or electrodes, propagation of the stimulation signals through the tissue, and stimulation parameters, such as stimulation amplitude, type, timing, etc.

Accordingly, cardiac rhythm management devices can include aspects located subcutaneously, though proximate the distal skin of the patient, as well as aspects, such as leads or electrodes, located near one or more organs of the patient. Separate from, or in addition to, the one or more electrodes or other sensors of the leads, the cardiac rhythm management device can include one or more electrodes or other sensors (e.g., a pressure sensor, an accelerometer, a gyroscope, a microphone, etc.) powered by a power source in the cardiac rhythm management device. The one or more electrodes or other sensors of the leads, the cardiac rhythm management device, or a combination thereof, can be configured to detect physiologic information from the patient, or provide one or more therapies or stimulation to the patient.

Implantable devices can additionally or separately include leadless cardiac pacemakers, small (e.g., smaller than traditional implantable cardiac rhythm management devices, in certain examples having a volume of about 1 cc, etc.), self-contained devices including one or more sensors, circuits, or electrodes configured to monitor physiologic information (e.g., heart rate, etc.) from, detect physiologic conditions (e.g., tachycardia) associated with, or provide one or more therapies or stimulation to the heart without traditional lead or implantable cardiac rhythm management device complications (e.g., required incision and pocket, complications associated with lead placement, breakage, or migration, etc.). In certain examples, leadless cardiac pacemakers can have more limited power and processing capabilities than a traditional cardiac rhythm management device; however, multiple leadless cardiac pacemaker devices can be implanted in or about the heart to detect physiologic information from, or provide one or more therapies or stimulation to, one or more chambers of the heart. The multiple leadless cardiac pacemaker devices can communicate between themselves, or one or more other implanted or external devices.

The implantable medical device 1002 can include a signal receiver circuit or an assessment circuit configured to detect or determine specific physiologic information of the patient 1001, or to determine one or more conditions or provide information or an alert to a user, such as the patient 1001 (e.g., a patient), a clinician, or one or more other caregivers or processes, such as described herein. The implantable medical device 1002 can alternatively or additionally be configured as a therapeutic device configured to treat one or more medical conditions of the patient 1001. The therapy can be delivered to the patient 1001 via the lead system and associated electrodes or using one or more other delivery mechanisms. The therapy can include delivery of one or more drugs to the patient 1001, such as using the implantable medical device 1002 or one or more of the other ambulatory medical devices, etc. In some examples, therapy can include cardiac resynchronization therapy for rectifying dyssynchrony and improving cardiac function in heart failure patients. In other examples, the implantable medical device 1002 can include a drug delivery system, such as a drug infusion pump to deliver drugs to the patient for managing arrhythmias or complications from arrhythmias, hypertension, hypotension, or one or more other physiologic conditions. In other examples, the implantable medical device 1002 can include one or more electrodes configured to stimulate the nervous system of the patient or to provide stimulation to the muscles of the patient airway, etc.

The wearable medical device 1003 can include one or more wearable or external medical sensors or devices (e.g., automatic external defibrillators (AEDs), Holter monitors, patch-based devices, smart watches, smart accessories, wrist-or finger-worn medical devices, such as a finger-based photoplethysmography sensor, etc.). The wearable medical device 1003 can include a signal receiver circuit or an assessment circuit configured to detect or determine specific physiologic information of the patient 1001, or to determine one or more conditions or provide information or an alert to a user, such as the patient 1001 (e.g., a patient), a clinician, or one or more other caregivers or processes, such as described herein.

The external system 1005 can include a dedicated hardware/software system, such as a programmer, a remote server-based patient management system, or alternatively a system defined predominantly by software running on a standard personal computer. The external system 1005 can manage the patient 1001 through the implantable medical device 1002 or one or more other ambulatory medical devices connected to the external system 1005 via a communication link 1011. In other examples, the implantable medical device 1002 can be connected to the wearable medical device 1003, or the wearable medical device 1003 can be connected to the external system 1005, via the communication link 1011. This can include, for example, programming or reprogramming the implantable medical device 1002 with different parameter settings to perform one or more of acquiring physiologic data, performing at least one self-diagnostic test (such as for a device operational status), analyzing the physiologic data, or optionally delivering or adjusting a therapy for the patient 1001. Additionally, the external system 1005 can send information to, or receive information from, the implantable medical device 1002 or the wearable medical device 1003 via the communication link 1011. Examples of the information can include real-time or stored physiologic data from the patient 1001, diagnostic data, such as detection of patient hydration status, hospitalizations, responses to therapies delivered to the patient 1001, or device operational status of the implantable medical device 1002 or the wearable medical device 1003 (e.g., battery status, lead impedance, etc.). The communication link 1011 can be an inductive telemetry link, a capacitive telemetry link, or a radio frequency (RF) telemetry link, such as a wireless telemetry based on, for example, Bluetooth® or IEEE 802.11 wireless fidelity “Wi-Fi” interfacing standards. Other configurations and combinations of patient data source interfacing are possible.

The external system 1005 can include an external device 1006 in proximity of the one or more ambulatory medical devices, and a remote device 1008 in a location relatively distant from the one or more ambulatory medical devices, in communication with the external device 1006 via a communication network 1007. Examples of the external device 1006 can include a medical device programmer. The external device 1006 or the remote device 1008 can be configured to evaluate collected device or patient information and provide alert notifications, among other possible functions. In an example, one or more of the external system 1005, the external device 1006, or the remote device 1008 can include a signal receiver circuit or an assessment circuit configured to receive or determine specific physiologic information of the patient 1001, such as from the implantable medical device 1002 or the wearable medical device 1003, or to determine one or more conditions or provide information or an alert to a user, such as the patient 1001 (e.g., a patient), a clinician, or one or more other caregivers or processes, such as described herein.

In an example, the remote device 1008 can include a centralized server acting as a central hub for collected data storage and analysis from a number of different sources. Combinations of information from the multiple sources can be used to make determinations and update individual patient health status or to adjust one or more alerts or determinations for one or more other patients. The server can be configured as a uni-, multi-, or distributed computing and processing system. The remote device 1008 can receive data from multiple patients. The data can be collected by the one or more ambulatory medical devices, among other data acquisition sensors or devices associated with the patient 1001. The server can include a memory device to store the data in a patient database. The server can include an alert analyzer circuit to evaluate the collected data to determine if specific alert condition is satisfied. Satisfaction of the alert condition may trigger a generation of alert notifications, such to be provided by one or more human-perceptible user interfaces. In some examples, the alert conditions may alternatively or additionally be evaluated by the one or more ambulatory medical devices, such as the implantable medical device. By way of example, alert notifications can include a Web page update, phone or pager call, E-mail, SMS, text, or “Instant” message, as well as a message to the patient and a simultaneous direct notification to emergency services and to the clinician. Other alert notifications are possible. The server can include an alert prioritizer circuit configured to prioritize the alert notifications. For example, an alert of a detected medical event can be prioritized using a similarity metric between the physiologic data associated with the detected medical event to physiologic data associated with the historical alerts.

In an example, similar to the alert notifications discussed above, the external system 1005 or one or more components thereof (e.g., the external device 1006, the remote device 1008, an assessment circuit, etc.) can be configured to schedule one or more follow-up appointments or adjust a schedule of one or more follow-up appointments for the patient such as in response to one or more alert notifications or other determinations, per a request of a clinician, etc.

The remote device 1008 may additionally include one or more locally configured clients or remote clients securely connected over the communication network 1007 to the server. Examples of the clients can include personal desktops, notebook computers, mobile devices, or other computing devices. System users, such as clinicians or other qualified medical specialists, may use the clients to securely access stored patient data assembled in the database in the server, and to select and prioritize patients and alerts for health care provisioning. In addition to generating alert notifications, the remote device 1008, including the server and the interconnected clients, may also execute a follow-up scheme by sending follow-up requests to the one or more ambulatory medical devices, or by sending a message or other communication to the patient 1001 (e.g., the patient), clinician or authorized third party as a compliance notification.

The communication network 1007 can provide wired or wireless interconnectivity. In an example, the communication network 1007 can be based on the Transmission Control Protocol/Internet Protocol (TCP/IP) network communication specification, although other types or combinations of networking implementations are possible. Similarly, other network topologies and arrangements are possible.

One or more of the external device 1006 or the remote device 1008 can output the detected medical events to a system user, such as the patient or a clinician, or to a process including, for example, an instance of a computer program executable in a microprocessor. In an example, the process can include an automated generation of a programming recommendation for an ambulatory medical device to optimize or improve patient condition or otherwise provide a desired clinical outcome. In an example, the external device 1006 or the remote device 1008 can include a respective display unit for displaying the physiologic or functional signals, or alerts, alarms, emergency calls, or other forms of warnings to signal the detection of one or more conditions. In some examples, the external system 1005 can include a signal receiver circuit and an assessment circuit, such as an external data processor configured to analyze the physiologic or functional signals received by the one or more ambulatory medical devices, and to confirm or reject one or more determinations made by one or more ambulatory medical devices, such as the implantable medical device 1002, the wearable medical device 1003, etc., or make additional determinations, etc. Computationally intensive algorithms, such as machine-learning algorithms, can be implemented in the external data processor.

With some examples, when parameter settings of an ambulatory medical device are analyzed using one or more trained machine learning models, and one or more differences between the parameter settings of the ambulatory medical device and the stored model parameter settings are detected, a recommendation to reprogram the medical device may be generated and presented to a clinician via a user interface of the remote device 1008, or via a user interface of a software application executing on a client device communicatively connected with the remote device 1008. The recommendation to reprogram the medical device may be determined by identifying differences between the parameter settings of the ambulatory medical device and the stored model parameter settings via the one or more machine learning models that otherwise went undetected by a clinician or a medical device programmer.

Portions of the one or more ambulatory medical devices or the external system 1005 can be implemented using hardware, software, firmware, or combinations thereof. Portions of the one or more ambulatory medical devices or the external system 1005 can be implemented using an application-specific circuit that can be constructed or configured to perform one or more functions or can be implemented using a general-purpose circuit that can be programmed or otherwise configured to perform one or more functions. Such a general-purpose circuit can include a microprocessor or a portion thereof, a microcontroller or a portion thereof, or a programmable logic circuit, a memory circuit, a network interface, and various components for interconnecting these components. For example, a “comparator” can include, among other things, an electronic circuit comparator that can be constructed to perform the specific function of a comparison between two signals or the comparator can be implemented as a portion of a general-purpose circuit that can be driven by a code instructing a portion of the general-purpose circuit to perform a comparison between the two signals. “Sensors” can include electronic circuits configured to receive information and provide an electronic output representative of such received information.

A therapy device 1010 can be configured to send information to or receive information from one or more of the ambulatory medical devices or the external system 1005 using the communication link 1011. In an example, the one or more ambulatory medical devices, the external device 1006, or the remote device 1008 can be configured to control one or more parameters of the therapy device 1010. The external system 1005 can allow for programming or reprogramming the one or more ambulatory medical devices and can receive information about one or more signals acquired by the one or more ambulatory medical devices, such as can be received via a communication link 1011. The external system 1005 can include a local external implantable medical device programmer. The external system 1005 can include a remote patient management system that can monitor patient health status or adjust one or more therapies such as from a remote location.

In certain examples, event storage can be triggered, such as received physiologic information or in response to one or more detected events or determined parameters meeting or exceeding a threshold (e.g., a static threshold, a dynamic threshold, or one or more other thresholds based on patient or population information, etc.). Information sensed or recorded in the high-power mode can be transitioned from short-term storage, such as in a loop recorder, to long-term or non-volatile memory, or in certain examples, prepared for communication to an external device separate from the medical device. In an example, cardiac electrical or cardiac mechanical information leading up to and in certain examples including the detected events can be stored, such as to increase the specificity of detection. In an example, multiple loop recorder windows (e.g., 2-minute windows, 4-minute windows, etc.) can be stored sequentially. In systems without early detection, to record this information, a loop recorder with a longer time period would be required at substantial additional cost (e.g., power, processing resources, component cost, amount of memory, etc.). Storing multiple windows using this early detection leading up to a single event can provide full event assessment with power and cost savings, in contrast to the longer loop recorder windows. In addition, the early detection can trigger additional parameter computation or storage, at different resolution or sampling frequency, without unduly taxing finite system resources.

In certain examples, one or more alerts can be provided, such as to the patient, to a clinician, or to one or more other caregivers (e.g., using a patient smart watch, a cellular or smart phone, a computer, etc.), in certain examples, in response to the transition to the high-power mode, in response to the detected event or condition, or after updating or transmitting information from a first device to a remote device. In other examples, the medical device itself can provide an audible or tactile alert to warn the patient of the detected condition. For example, the patient can be alerted in response to a detected condition so they can engage in corrective action, such as sitting down, etc.

In certain examples, a therapy can be provided in response to the detected condition. For example, a pacing therapy can be provided, enabled, or adjusted, such as to disrupt or reduce the impact of the detected event. In other examples, delivery of one or more drugs (e.g., a vasoconstrictor, pressor drugs, etc.) can be triggered, provided, or adjusted, such as using a drug pump, in response to the detected condition, alone or in combination with a pacing therapy, such as that described above, for example, to increase arterial pressure, to maintain cardiac output, to disrupt or reduce the impact of the detected event, or combinations thereof.

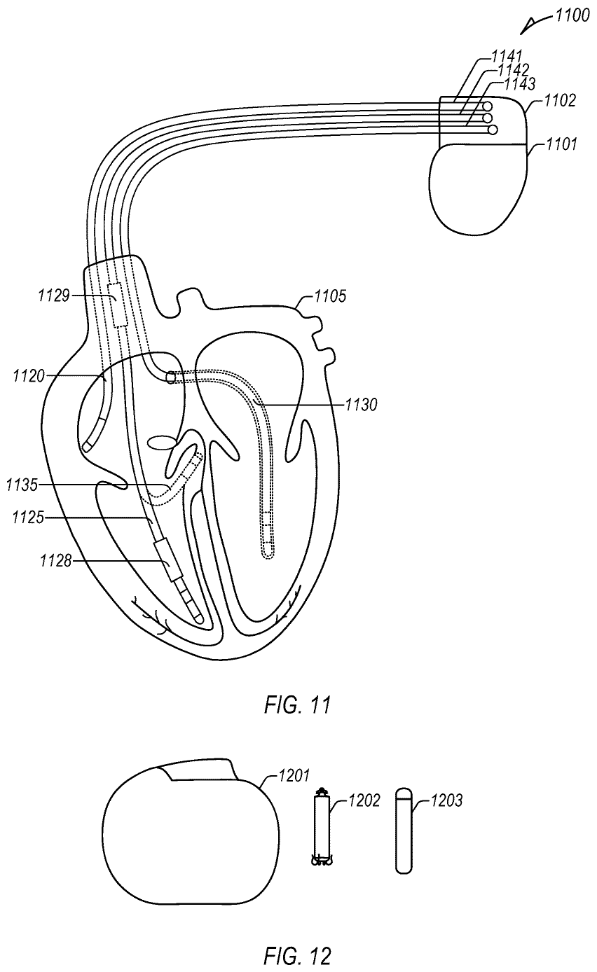

FIG. 11 illustrates an example implantable medical device system 1100 including an implantable medical device 1101 electrically coupled to a heart 1105, such as through one or more leads coupled to the implantable medical device 1101 through one or more lead ports, including first, second, or third lead ports 1141, 1142, 1143 in a header 1102 of the implantable medical device 1101. In an example, the implantable medical device 1101 can include an antenna, such as in the header 1102, configured to enable communication with an external system and one or more electronic circuits (e.g., an assessment circuit, etc.) in a hermetically sealed housing (CAN).

The implantable medical device 1101 may include an insertable cardiac monitor, pacemaker, defibrillator, cardiac resynchronization therapy device, or other subcutaneous implantable medical device or cardiac rhythm management device configured to be implanted in a chest of a patient, having one or more leads to position one or more electrodes or other sensors at various locations in or near the heart 1105, such as in one or more of the atria or ventricles. Separate from, or in addition to, the one or more electrodes or other sensors of the leads, the implantable medical device system 1100 can include one or more electrodes or other sensors (e.g., a pressure sensor, an accelerometer, a gyroscope, a microphone, etc.) powered by a power source in the implantable medical device 1101. The one or more electrodes or other sensors of the leads, the implantable medical device 1101, or a combination thereof, can be configured to detect physiologic information from, or provide one or more therapies or stimulation to, the patient.

Implantable devices can additionally include a leadless cardiac pacemaker, small (e.g., smaller than traditional implantable devices, in certain examples having a volume of about 1 cc, etc.), self-contained devices including one or more sensors, circuits, or electrodes configured to monitor physiologic information (e.g., heart rate, etc.) from, detect physiologic conditions (e.g., tachycardia) associated with, or provide one or more therapies or stimulation to the heart 1105 without traditional lead or implantable device complications (e.g., required incision and pocket, complications associated with lead placement, breakage, or migration, etc.). In certain examples, a leadless cardiac pacemaker can have more limited power and processing capabilities than a traditional CRM device; however, multiple leadless cardiac pacemaker devices can be implanted in or about the heart to detect physiologic information from, or provide one or more therapies or stimulation to, one or more chambers of the heart. The multiple leadless cardiac pacemaker devices can communicate between themselves, or one or more other implanted or external devices.

The implantable medical device 1101 can include one or more electronic circuits configured to sense one or more physiologic signals, such as an electrogram or a signal representing mechanical function of the heart 1105. In certain examples, the housing may function as an electrode such as for sensing or pulse delivery. For example, an electrode from one or more of the leads may be used together with the housing such as for unipolar sensing of an electrogram or for delivering one or more pacing pulses. A defibrillation electrode (e.g., the first defibrillation coil electrode 1128, the second defibrillation coil electrode 1129, etc.) may be used together with the housing to deliver one or more cardioversion/defibrillation pulses.

In an example, the implantable medical device 1101 can sense impedance such as between electrodes located on one or more of the leads or the housing. The implantable medical device 1101 can be configured to inject current between a pair of electrodes, sense the resultant voltage between the same or different pair of electrodes, and determine impedance, such as using Ohm's Law. The impedance can be sensed in a bipolar configuration in which the same pair of electrodes can be used for injecting current and sensing voltage, a tripolar configuration in which the pair of electrodes for current injection and the pair of electrodes for voltage sensing can share a common electrode, or tetrapolar configuration in which the electrodes used for current injection can be distinct from the electrodes used for voltage sensing, etc. In an example, the implantable medical device 1101 can be configured to inject current between an electrode on one or more of the first, second, third, or fourth leads 1120, 1125, 1130, 1135 and the housing, and to sense the resultant voltage between the same or different electrodes and the housing.

The implantable medical device 1101 can integrate one or more other physiologic sensors to sense one or more other physiologic signals, such as one or more of heart rate, heart rate variability, thoracic or intrathoracic impedance, intracardiac impedance, arterial pressure, pulmonary artery pressure, right ventricular pressure, left ventricular coronary pressure, coronary blood temperature, blood oxygen saturation, one or more heart sounds, physical activity or exertion level, physiologic response to activity, posture, respiration, body weight, or body temperature. The arrangement and functions of these leads and electrodes are described above by way of example and not by way of limitation. Depending on the need of the patient and the capability of the implantable device, other arrangements and uses of these leads and electrodes are contemplated.

FIG. 12 illustrates example implantable medical devices, including a subcutaneous implantable cardioverter defibrillator 1201, a leadless cardiac pacemaker 1202 including tines for placement, and an insertable cardiac monitor 1203.

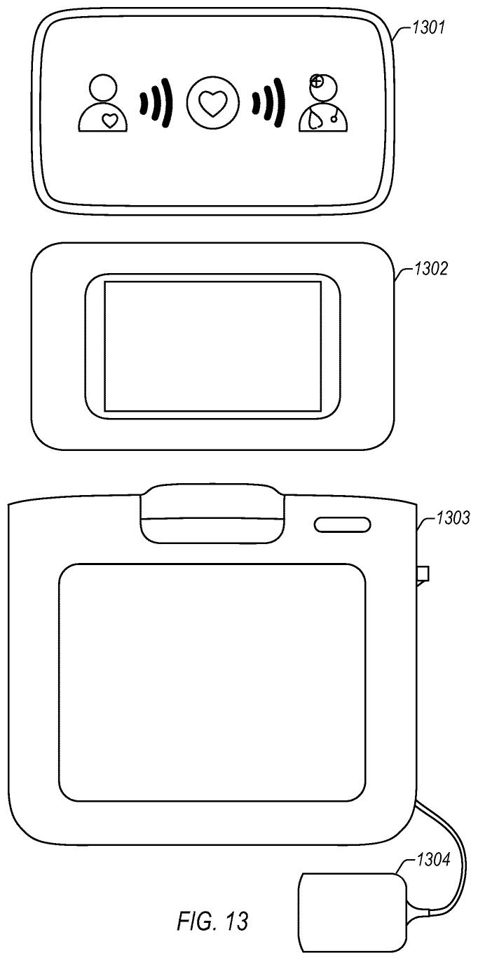

FIG. 13 illustrates example remote patient management systems for communication with an ambulatory medical device, such as for receiving information from or providing information to, including programming instructions, one or more ambulatory medical devices. The example remote patient management systems include a first remote patient management device 1301 (e.g., a LATITUDE™ NXT Remote Patient Management System) for at-home monitoring and RF telemetry capabilities through one or more communication circuits with an ambulatory medical device and communication to a cloud-based server or clinician programming environment through a network connection, a second remote patient management device 1302 (e.g., an EMBLEM™ S-ICD Programmer) with RF telemetry capabilities through one or more communication circuits and an optional external telemetry wand for communication with an ambulatory medical device, and a third remote patient management system 1303 (e.g., a LATITUDE™ Programming System, Model 3300, etc.) with RF telemetry capabilities through one or more communication circuits and an external telemetry wand 1304 (e.g., a Model 6395 Telemetry Wand, etc.) including an external telemetry coil configured for inductive communication with a corresponding telemetry coil of an implantable medical device. Although not illustrated herein, the remote patient management systems can include one or more other remote patient management systems, such as one or more other LATITUDE™ Programming systems, a remote patient monitoring application for a mobile device of a patient or other caregiver, etc. Each type of remote patient monitoring or management system has different capabilities and in certain examples permissions with respect to different programming instructions or features.

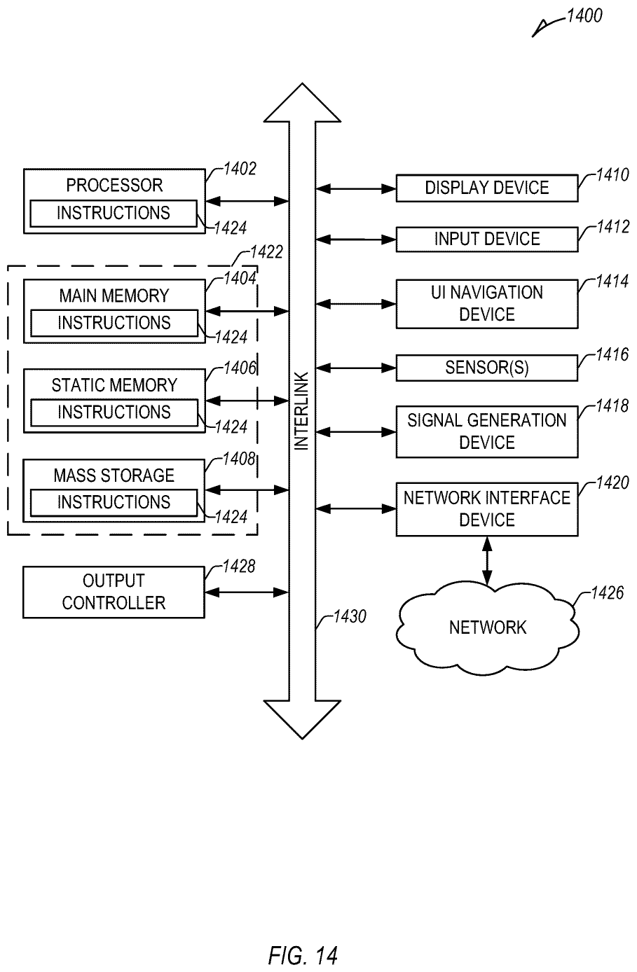

FIG. 14 illustrates a block diagram of an example machine 1400 upon which any one or more of the techniques (e.g., methodologies) discussed herein may perform. Portions of this description may apply to the computing framework of one or more of the medical devices described herein, such as the implantable medical device, the external programmer, etc. Further, as described herein with respect to medical device components, systems, or machines, such may require regulatory-compliance not capable by generic computers, components, or machinery.

Examples, as described herein, may include, or may operate by, logic or a number of components, or mechanisms in the machine 1400. Circuitry (e.g., processing circuitry, an assessment circuit, etc.) is a collection of circuits implemented in tangible entities of the machine 1400 that include hardware (e.g., simple circuits, gates, logic, etc.). Circuitry membership may be flexible over time. Circuitries include members that may, alone or in combination, perform specified operations when operating. In an example, hardware of the circuitry may be immutably designed to perform a specific operation (e.g., hardwired). In an example, the hardware of the circuitry may include variably connected physical components (e.g., execution units, transistors, simple circuits, etc.) including a machine-readable medium physically modified (e.g., magnetically, electrically, moveable placement of invariant massed particles, etc.) to encode instructions of the specific operation. In connecting the physical components, the underlying electrical properties of a hardware constituent are changed, for example, from an insulator to a conductor or vice versa. The instructions enable embedded hardware (e.g., the execution units or a loading mechanism) to create members of the circuitry in hardware via the variable connections to perform portions of the specific operation when in operation. Accordingly, in an example, the machine-readable medium elements are part of the circuitry or are communicatively coupled to the other components of the circuitry when the device is operating. In an example, any of the physical components may be used in more than one member of more than one circuitry. For example, under operation, execution units may be used in a first circuit of a first circuitry at one point in time and reused by a second circuit in the first circuitry, or by a third circuit in a second circuitry at a different time. Additional examples of these components with respect to the machine 1400 follow.

In alternative embodiments, the machine 1400 may operate as a standalone device or may be connected (e.g., networked) to other machines. In a networked deployment, the machine 1400 may operate in the capacity of a server machine, a client machine, or both in server-client network environments. In an example, the machine 1400 may function as a peer machine in peer-to-peer (P2P) (or other distributed) network environment. The machine 1400 may be a personal computer (PC), a tablet PC, a set-top box (STB), a personal digital assistant (PDA), a mobile telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations.

The machine 1400 (e.g., computer system) may include a hardware processor 1402 (e.g., a central processing unit (CPU), a graphics processing unit (GPU), a hardware processor core, or any combination thereof), a main memory 1404, a static memory 1406 (e.g., memory or storage for firmware, microcode, a basic-input-output (BIOS), unified extensible firmware interface (UEFI), etc.), and mass storage 1408 (e.g., hard drive, tape drive, flash storage, or other block devices) some or all of which may communicate with each other via an interlink 1430 (e.g., bus). The machine 1400 may further include a display unit 1410, an alphanumeric input device 1412 (e.g., a keyboard), and a user interface (UI) navigation device 1414 (e.g., a mouse). In an example, the display unit 1410, input device 1412, and UI navigation device 1414 may be a touch screen display. The machine 1400 may additionally include a signal generation device 1418 (e.g., a speaker), a network interface device 1420, and one or more sensors 1416, such as a global positioning system (GPS) sensor, compass, accelerometer, or one or more other sensors. The machine 1400 may include an output controller 1428, such as a serial (e.g., universal serial bus (USB), parallel, or other wired or wireless (e.g., infrared (IR), near field communication (NFC), etc.) connection to communicate or control one or more peripheral devices (e.g., a printer, card reader, etc.).

Registers of the processor 1402, the main memory 1404, the static memory 1406, or the mass storage 1408 may be, or include, a machine-readable medium 1422 on which is stored one or more sets of data structures or instructions 1424 (e.g., software) embodying or utilized by any one or more of the techniques or functions described herein. The instructions 1424 may also reside, completely or at least partially, within any of registers of the processor 1402, the main memory 1404, the static memory 1406, or the mass storage 1408 during execution thereof by the machine 1400. In an example, one or any combination of the hardware processor 1402, the main memory 1404, the static memory 1406, or the mass storage 1408 may constitute the machine-readable medium 1422. While the machine-readable medium 1422 is illustrated as a single medium, the term “machine-readable medium” may include a single medium or multiple media (e.g., a centralized or distributed database, and/or associated caches and servers) configured to store the one or more instructions 1424.

The term “machine-readable medium” may include any medium that is capable of storing, encoding, or carrying instructions for execution by the machine 1400 and that cause the machine 1400 to perform any one or more of the techniques of the present disclosure, or that is capable of storing, encoding, or carrying data structures used by or associated with such instructions. Non-limiting machine-readable medium examples may include solid-state memories, optical media, magnetic media, and signals (e.g., radio frequency signals, other photon-based signals, sound signals, etc.). In an example, a non-transitory machine-readable medium comprises a machine-readable medium with a plurality of particles having invariant (e.g., rest) mass, and thus are compositions of matter. Accordingly, non-transitory machine-readable media are machine-readable media that do not include transitory propagating signals. Specific examples of non-transitory machine-readable media may include: non-volatile memory, such as semiconductor memory devices (e.g., Electrically Programmable Read-Only Memory (EPROM), Electrically Erasable Programmable Read-Only Memory (EEPROM)) and flash memory devices; magnetic disks, such as internal hard disks and removable disks; magneto-optical disks; and CD-ROM and DVD-ROM disks.

The instructions 1424 may be further transmitted or received over a communications network 1426 using a transmission medium via the network interface device 1420 utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), mobile telephone networks (e.g., cellular networks), Plain Old Telephone (POTS) networks, and wireless data networks (e.g., Institute of Electrical and Electronics Engineers (IEEE) 802.11 family of standards known as Wi-Fi®, IEEE 802.16 family of standards known as WiMax®), IEEE 802.15.4 family of standards, peer-to-peer (P2P) networks, among others. In an example, the network interface device 1420 may include one or more physical jacks (e.g., Ethernet, coaxial, or phone jacks) or one or more antennas to connect to the communications network 1426. In an example, the network interface device 1420 may include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding, or carrying instructions for execution by the machine 1400, and includes digital or analog communications signals or other intangible medium to facilitate communication of such software. A transmission medium is a machine-readable medium.

Various embodiments are illustrated in the figures above. One or more features from one or more of these embodiments may be combined to form other embodiments. Method examples described herein can be machine or computer-implemented at least in part. Some examples may include a computer-readable medium or machine-readable medium encoded with instructions operable to configure an electronic device or system to perform methods as described in the above examples. An implementation of such methods can include code, such as microcode, assembly language code, a higher-level language code, or the like. Such code can include computer readable instructions for performing various methods. The code can form portions of computer program products. Further, the code can be tangibly stored on one or more volatile or non-volatile computer-readable media during execution or at other times.

The above detailed description is intended to be illustrative, and not restrictive. The scope of the disclosure should, therefore, be determined with references to the appended claims, along with the full scope of equivalents to which such claims are entitled.

What is claimed is:

1. A medical device system for improving posture detection to optimize medical device system resource allocation, comprising:
a signal receiver circuit configured to receive posture information of a patient from a posture sensor of an ambulatory medical device, the posture information including posture information from multiple axes with respect to the posture sensor of the ambulatory medical device; and
an assessment circuit configured to determine posture information for the patient using the received posture information, including to:
cluster the received posture information occurring over a first time period into at least two clusters, wherein one of the at least two clusters includes a supine or upright cluster and at least one other cluster;
determine, for each respective cluster, an opposite density measure indicative of a density of the posture information opposite the respective cluster in multi-dimensional space with respect to the posture sensor;
identify the supine or upright cluster using the opposite density measures of the at least two clusters; and
determine the posture information for the patient using the identified supine or upright cluster.

2. The medical device system of claim 1, wherein the supine or upright cluster includes a supine/upright cluster,
wherein the assessment circuit is configured to identify the supine/upright cluster as the respective cluster having a lowest of the determined opposite density measure.

3. The medical device system of claim 1, wherein the supine or upright cluster includes the upright cluster,
wherein the assessment circuit is configured to identify the upright cluster as the respective cluster having a lowest of the determined opposite density measure.

4. The medical device system of claim 1, wherein the at least one other cluster includes a first side cluster and a second side cluster,
wherein the assessment circuit is configured to identify a left side cluster and a right side cluster using the first side cluster, the second side cluster, and the supine or upright cluster.

5. The medical device system of claim 1, wherein the posture sensor of the ambulatory medical device includes a three-dimensional accelerometer,
wherein the posture information includes three-dimensional acceleration information with respect to the accelerometer of the ambulatory medical device,
wherein the assessment circuit is configured to determine, for each respective cluster, the opposite density measure indicative of the density of the three-dimensional acceleration information occurring over the first time period opposite the respective cluster.

6. The medical device system of claim 5, wherein the assessment circuit is configured to separate the supine or upright cluster into separate supine and upright clusters based on a spread of the three-dimensional acceleration information in the supine or upright cluster,
wherein the spread of the supine cluster is less than the spread of the upright cluster.

7. The medical device system of claim 6, wherein the three-dimensional acceleration information is with respect to UVW space of the accelerometer of the ambulatory medical device,
wherein the assessment circuit is configured to:
determine centroids of at least three clusters; and
transform the three-dimensional acceleration information from the UVW space of the accelerometer into XYZ space of the patient using the determined centroids of the at least three clusters.

8. The medical device system of claim 7, wherein to determine the centroids of the at least three clusters includes to determine centroids of the supine cluster, a first or left side cluster, and a second or right side cluster.

9. The medical device system of claim 7, wherein the assessment circuit is configured to label the three-dimensional acceleration information and to determine posture trends for the patient over the first time period using the labeled information.

10. The medical device system of claim 9 wherein the assessment circuit is configured to determine a patient health status using changes in the determined posture trends.

11. The medical device system of claim 9, wherein the assessment circuit is configured to provide an output of the determined posture trends to a user interface for display to a user or to another circuit to control or adjust a process or function of the medical device system.

12. The medical device system of claim 9, wherein the assessment circuit is configured to compare the determined posture trends for the patient over the first time period to determined posture trends for the patient over one or more other time periods and to provide an alert if the determined posture trends for the patient over the first time period exceed the determined posture trends for the patient over the one or more other time periods by more than a threshold amount.

13. The medical device system of claim 9, wherein the assessment circuit is configured to alter or adjust one or more modes or functions of the ambulatory medical device or the medical device system based on the determined posture trends for the patient over the first time period,
wherein the one or more modes or functions includes at least one of: an active state of a sensor of the medical device system, a sampling frequency or resolution of a sensor of the medical device system, an amount of data storage of the ambulatory medical device or the medical device system, or a time or amount of communication of stored information outside of the ambulatory medical device.

14. A method for improving posture detection to optimize medical device system resource allocation, comprising:
receiving acceleration information of a patient from an accelerometer of an ambulatory medical device, the acceleration information including three-dimensional acceleration information with respect to the accelerometer of the ambulatory medical device; and
determining posture information for the patient using the received acceleration information, including:
clustering the received acceleration information occurring over a first time period into at least two clusters, wherein one of the at least two clusters includes a supine or upright cluster and at least one other cluster;
determining, for each respective cluster, an opposite density measure indicative of a density of the three-dimensional acceleration information opposite the respective cluster;
identifying the supine or upright cluster using the opposite density measures of the at least two clusters; and
determining the posture information for the patient using the identified supine or upright cluster.

15. The method of claim 14, wherein the supine or upright cluster includes a supine/upright cluster,
wherein identifying the supine or upright cluster includes identifying the supine/upright cluster as the respective cluster having a lowest of the determined opposite density measure.

16. The method of claim 14, wherein the supine or upright cluster includes the upright cluster,
wherein identifying the supine or upright cluster includes identifying the upright cluster as the respective cluster having a lowest of the determined opposite density measure.

17. The method of claim 14, wherein the at least one other cluster includes a first side cluster and a second side cluster,
wherein determining the posture information for the patient includes identifying a left side cluster and a right side cluster using the first side cluster, the second side cluster, and the supine or upright cluster.

18. The method of claim 14, wherein determining the posture information for the patient includes:
separating the supine or upright cluster into separate supine and upright clusters based on a spread of the three-dimensional acceleration information in the supine or upright cluster,
wherein the spread of the supine cluster is less than the spread of the upright cluster.

19. The method of claim 18, wherein the three-dimensional acceleration information is with respect to UVW space of the accelerometer of the ambulatory medical device,
wherein determining the posture information for the patient includes:
determining centroids of at least three clusters, including the supine cluster, a first or left side cluster, and a second or right side cluster; and
transforming the three-dimensional acceleration information from the UVW space of the accelerometer into XYZ space of the patient using the determined centroids of the at least three clusters.

20. The method of claim 18, wherein determining the posture information for the patient includes:
labeling the received acceleration information;
determining posture trends for the patient over the first time period using the labeled information; and
determining a patient health status using changes in the determined posture trends.
